(19) United States
(12) Patent Application Publication
(10) Pub. No.: US 2022/0003769 A1
(43) Pub. Date: Jan. 6, 2022

(54) Method Of Detecting Lung Cancer

(71) Applicant: BioMark Cancer Systems Inc., Richmond (CA)

(72) Inventor: Rashid Bux, British Columbia (CA)

(73) Assignee: BioMark Cancer Systems Inc., Richmond, BC (CA)

(21) Appl. No.: 17/415,601

(22) PCT Filed: Dec. 23, 2019
(86) PCT No.: PCT/CA2019/051908

§ 371 Date: Jun. 17, 2021

Related U.S. Application Data
(60) Provisional application No. 62/784,365, filed on Dec. 21, 2018.

(51) Int. Cl. G01N 33/574

(52) U.S. Cl. CPC G01N 33/57423

(57) ABSTRACT

A biomarker panel for a serum test for detecting lung cancer, wherein the biomarkers are selected from the group of biomarkers consisting of arginine, C18.2, decadienylcamitine (C10:2), LYSOC18.2, methionine, ornithine, PC32:2AA, PC36.0AA, PC36.0AE, putrescine, spermidine, spermine, and valine. The serum test for diagnosing lung cancer may account for smoking history.

### TECHNICAL FIELD

The present disclosure relates to a method of detecting cancer and, in particular, to a method of detecting lung cancer by measuring polyamine metabolites and other metabolites.

### BACKGROUND

The polyamine pathway has been demonstrated to be significantly up-regulated in cancer cells. Spermidine/spermine N1-acetyltransferase (SSAT) is recognized as a critical enzyme in the pathway and is highly regulated in all mammalian cells. While SSAT is present in normal tissues in very low concentrations, it is present at much higher levels in cancer cells. Therefore, as cellular levels of SSAT increase, measurement of its enzymatic activity correlates with the presence and severity of cancer.

International Patent Application Publication No. WO 2016/205960 A1, which was published in the name of BioMark Cancer Systems Inc. on Dec. 29, 2016, discloses a biomarker panel for a urine test for detecting lung cancer in which the biomarker panel detects a biomarker selected from the group of biomarkers consisting of DMA, C5:1, C10:1, ADMA, C5-OH, SDMA, and kynurenine, or a combination thereof. There is also disclosed a biomarker panel for a serum test for detecting lung cancer in which the biomarker panel detects a biomarker selected from the group of biomarkers consisting of valine, arginine, ornithine, methionine, spermidine, spermine, diacetylspermine, C10:2, PC aa C32:2, PC ae C36:0, and PC ae C44:5; and lysoPC a C18:2, or a combination thereof.

### SUMMARY

Disclosed herein is a biomarker panel for a serum test for detecting lung cancer, wherein the biomarkers are selected from the group of biomarkers consisting of arginine, C18.2, decadienylcarnitine (C10:2), LYSOC18.2, methionine, ornithine, PC32:2AA, PC36.0AA, PC36.0AE, putrescine, spermidine, spermine, and valine. The serum test for diagnosing lung cancer may account for smoking history.

The biomarker panel may be used to diagnose stage 1 lung cancer. The biomarker panel may be used to diagnose stage 2 lung cancer. The biomarker panel may be used to differentiate between stage 1 adenocarcinoma lung cancer and stage 1 squamous lung cancer. The biomarker panel may be used to differentiate between stage 2 adenocarcinoma lung cancer and stage 2 squamous lung cancer. The biomarker panel may be used to diagnose combined stage 1 and 2 adenocarcinoma lung cancer. The biomarker panel may be used to diagnose combined stage 1 and 2 squamous lung cancer. The biomarker panel may be used to diagnose combined stage 1 adenocarcinoma lung cancer and squamous lung cancer. The biomarker panel may be used to diagnose combined stage 2 adenocarcinoma lung cancer and squamous lung cancer. The biomarker panel may be used to diagnose late stage 3b/4 lung cancer.

### BRIEF DESCRIPTION OF THE DRAWINGS

FIG. 1 is a variable importance in projection (VIP) plot ranking discriminating serum metabolites in descending order of importance based on a partial least squares discriminant analysis (PLS-DA) showing separation between control patients and stage 1 lung cancer patients;

FIG. 2 is an area under the receiver operating characteristic curve (AUROC) including the three most important serum metabolites from the VIP plot shown in FIG. 1;

FIG. 3 is an AUROC curve including the six most important serum metabolites from the VIP plot of FIG. 1;

FIG. 4 is an AUROC curve including smoking status and the three most important serum metabolites from the VIP plot shown in FIG. 1;

FIG. 5 is an AUROC curve including smoking status, body mass index and the three most important serum metabolites from the VIP plot shown in FIG. 1;

FIG. 6 is a VIP plot ranking discriminating serum metabolites in descending order of importance based on a PLS-DA analysis showing separation between control patients and stage 2 lung cancer patients;

FIG. 7 is an AUROC curve including the two most important serum metabolites from the VIP plot shown in FIG. 6;

FIG. 8 is an AUROC curve including the seven most important serum metabolites from the VIP plot shown in FIG. 6;

FIG. 9 is an AUROC curve including smoking status and the three most important serum metabolites from the VIP plot shown in FIG. 6;

FIG. 10 is an AUROC curve including smoking status, body mass index and the three most important serum metabolites from the VIP plot shown in FIG. 6;

FIG. 11 is a VIP plot ranking discriminating serum metabolites in descending order of importance based on a PLS-DA analysis showing separation between stage 1 adenocarcinoma lung cancer patients and stage 1 squamous lung cancer patients;

FIG. 12 is an AUROC curve including the four most important serum metabolites from the VIP plot shown in FIG. 11;

FIG. 13 is an AUROC curve including smoking status and the four most important serum metabolites from the VIP plot shown in FIG. 11;

FIG. 14 is a VIP plot ranking discriminating serum metabolites in descending order of importance based on a PLS-DA analysis showing separation between stage 2 adenocarcinoma lung cancer patients and stage 2 squamous lung cancer patients;

FIG. 15 is an AUROC curve including the four most important serum metabolites from the VIP plot shown in FIG. 14;

FIG. 16 is an AUROC curve including the seven most important serum metabolites from the VIP plot shown in FIG. 14;

FIG. 17 is an AUROC curve including smoking status and the four most important serum metabolites from the VIP plot shown in FIG. 14;

FIG. 18 is a principal component analysis (PCA) plot of Groups 1-9 patients vs. control patients;

FIG. 19 is another PCA plot of Groups 1-9 patients vs. control patients;

FIG. 20 is a partial least square discriminant analysis (PLS-DA) plot of Groups 1-9 patients vs. control patients;

FIG. 21 is a dendrogram of samples of Groups 1-9 patients vs. control patients;

FIG. 22 is a PCA plot of Groups 1-9 patients vs. control patients after cleaning data;

FIG. 23 is another PCA plot of Groups 1-9 patients vs. control patients after cleaning data;

FIG. 24 is a PLS-DA plot of Groups 1-9 patients vs. control patients after cleaning data;

FIG. 25 is another PLS-DA plot of Groups 1-9 patients vs. control patients after cleaning data;

FIG. 26 is a dendrogram of samples of Groups 1-9 patients vs. control patients after cleaning data.;

FIG. 27 is an AUROC curve including period of smoking for total lung cancer patients (Groups 1-6) vs. control patients;

FIG. 28 is an AUROC curve including cigarette consumption (pack/year) for total lung cancer patients (Groups 1-6) vs. control patients;

FIG. 29 is an AUROC curve including smoking status (yes/no) for total lung cancer patients (Groups 1-6) vs. control patients;

FIG. 30 is an AUROC curve including age for total lung cancer patients (Groups 1-6) vs. control patients;

FIG. 31 is an AUROC curve including BMI for total lung cancer patients (Groups 1-6) vs. control patients;

FIG. 32 is an AUROC curve including gender for total lung cancer patients (Groups 1-6) vs. control patients;

FIG. 33 is an AUROC curve including metabolites only for total lung cancer patients (Groups 1-6) vs. control patients;

FIGS. 34A and 34B are show data distribution and concentration range of metabolites for total lung cancer patients (Groups 1-6) vs. control patients;

FIG. 35 is a PCA plot of combined stage 1 and 2 adenocarcinoma lung cancer patients vs. control patients;

FIG. 36 is an AUROC curve including metabolites only for combined stage 1 and 2 adenocarcinoma lung cancer patients vs. control patients;

FIG. 37 is an AUROC curve including metabolites and BMI for combined stage 1 and 2 adenocarcinoma lung cancer patients vs. control patients;

FIGS. 38A and 38B show data distribution and concentration range of metabolites for combined stage 1 and 2 adenocarcinoma lung cancer patients vs. control patients;

FIG. 39 is a PCA plot of combined stage 1 and 2 squamous lung cancer patients vs. control patients;

FIG. 40 is an AUROC curve including metabolites only for combined stage 1 and 2 squamous lung cancer patients vs. control patients;

FIG. 41 is an AUROC curve including metabolites and BMI for combined stage 1 and 2 squamous lung cancer patients vs. control patients;

FIG. 42 is an AUROC curve including metabolites and smoking for combined stage 1 and 2 squamous lung cancer patients vs. control patients;

FIGS. 43A and 43B show data distribution and concentration range of metabolites for combined stage 1 and 2 squamous lung cancer patients vs. control patients;

FIG. 44 is a PCA plot of stage 3b/4 NSCLC lung cancer patients (Group 5) vs. control patients;

FIG. 45 is an AUROC curve including metabolites only for stage 3b/4 NSCLC lung cancer patients (Group 5) vs. control patients;

FIG. 46 is an AUROC curve including metabolites and BMI for stage 3b/4 NSCLC lung cancer patients (Group 5) vs. control patients;

FIG. 47 is an AUROC curve including metabolites and smoking status for stage 3b/4 NSCLC lung cancer patients (Group 5) vs. control patients;

FIGS. 48A and 48B show data distribution and concentration range of metabolites for stage 3b/4 NSCLC lung cancer patients (Group 5) vs. control patients;

FIG. 49 is a PCA plot of combined stage 1 (adenocarcinoma and squamous) lung cancer patients vs. control patients;

FIG. 50 is a PLS-DA plot of combined stage 1 (adenocarcinoma and squamous) lung cancer patients vs. control patients;

FIG. 51 is an AUROC curve including metabolites, cigarette consumption and period of smoking for combined stage 1 (adenocarcinoma and squamous) lung cancer patients vs. control patients;

FIG. 52 is an AUROC curve including metabolites, cigarette consumption, period of smoking and BMI for combined stage 1 (adenocarcinoma and squamous) lung cancer patients vs. control patients;

FIG. 53 is an AUROC curve including metabolites and smoking status for combined stage 1 (adenocarcinoma and squamous) lung cancer patients vs. control patients;

FIG. 54 is an AUROC curve including metabolites only for stage 1 (adenocarcinoma and squamous) lung cancer patients vs. control patients;

FIG. 55 is an AUROC curve including metabolites and BMI for combined stage 1 (adenocarcinoma and squamous) lung cancer patients vs. control patients;

FIG. 56 is a PCA plot of combined stage 2 (adenocarcinoma and squamous) lung cancer patients vs. control patients;

FIG. 57 is a PLS-DA plot of combined stage 2 (adenocarcinoma and squamous) lung cancer patients vs. control patients;

FIG. 58 is an AUROC curve including metabolites only for combined stage 2 (adenocarcinoma and squamous) lung cancer patients vs. control patients;

FIG. 59 is an AUROC curve including metabolites and BMI for combined stage 2 (adenocarcinoma and squamous) lung cancer patients vs. control patients;

FIG. 60 is an AUROC curve including metabolites and period of smoking for combined stage 2 (adenocarcinoma and squamous) lung cancer patients vs. control patients;

FIG. 61 is an AUROC curve including metabolites and cigarette consumption for combined stage 2 (adenocarcinoma and squamous) lung cancer patients vs. control patients; and

FIG. 62 is an AUROC curve including metabolites and smoking status for combined stage 2 (adenocarcinoma and squamous) lung cancer patients vs. control patients.

### DESCRIPTION OF SPECIFIC EMBODIMENTS

Serum samples collected from 60 control patients and 197 lung cancer patients were analyzed using a combination of direct injection mass spectrometry and reverse-phase LC-MS/MS. An AbsoluteIDQ® p180 Kit obtained from Biocrates Life Sciences AG of Eduard-Bodem-Gasse 8 6020, Innsbruck, Austria was used in combination with an API4000 Qtrap® tandem mass spectrometer obtained from Applied Biosystems/MDS Sciex of 850 Lincoln Centre Drive, Foster City, Calif., 94404, United States of America, for the targeted identification and quantification of up to 180 different endogenous metabolites including amino acids, acylcarnitines, biogenic amines, glycerophospholipids, sphingolipids and sugars. Table 1 shows the clinical characteristics of the control patients and lung cancer patients.

TABLE 1
Clinical characteristics of control patients and lung
cancer patients.

| | | Number of | After |
|---|---|---|---|
| Group | Request | Samples | Cleaning |
| 1 | 50 stage 1 NSCLC adenocarcinoma | 50 | 36 |
| 2 | 20 stage 1 NSCLC squamous | 20 | 20 |
| 3 | 40 stage 2 NSCLC adenocarcinoma | 40 | 35 |
| 4 | 20 stage 2 NSCLC squamous | 20 | 20 |
| 5 | 50 stage 3b/4 NSCLC adenocarcinoma | 26 | 26 |
| | and squamous | | |
| 6 | 25 patients at various stages of SLCL | 14 | 14 |
| | (smokers and non-smokers) | | |
| 7 | 5 patients with mesothelioma | 7 | 4 |
| 8 | 10 patients with COPD (5 with cancer, 5 | 10 | 10 |
| | without cancer) | | |
| 9 | TB, 5 asthma, 5 bronchiectasis | 10 | 6 |
| 10 | 60 healthy control subjects (20 smokers | 60 | 37 |
| | or 40 non-smokers) | | |

| | | |
|---|---|---|
| Total | 257 | 208 |

The following metabolites were analyzed in the serum samples: valine, putrescience, MTA, arginine, ornithine, spermidine, spermine, di-acetyl spermine, methionine, decadienylcarnitine (C10:2), PC aa C32:2, PC aa C36:0, PC ae C36:0, lysoPC a C18:2. Metabolites with more than 20% of missing values in all the groups were removed. A large number of the missing values came from being below the limit of detection. Two metabolites, MTA and di-acetyl spermine, were removed due to high missing values. If missing values were less than 20%, the missing values were imputed by half of the minimum value for that metabolite. The total number of metabolites analyzed was 13.

The method used combines the derivatization and extraction of analytes, and the selective mass-spectrometric detection using multiple reaction monitoring (MRM) pairs. Isotope-labeled internal standards and other internal standards are integrated in an AbsoluteIDQ® p180 Kit plate filter for metabolite quantification. The AbsoluteIDQ® p180 Kit contains a 96 deep-well plate with a filter plate attached with sealing tape as well as reagents and solvents used to prepare the plate assay. First 14 wells in the AbsoluteIDQ® p180 Kit were used for one blank, three zero samples, seven standards and three quality control samples provided with each AbsoluteIDQ® p180 Kit. All the serum samples were analyzed with the AbsolutelDQ p180 Kit using the protocol described in the AbsoluteIDQ® p180 Kit User Manual.

Serum samples were thawed on ice and were vortexed and centrifuged at 2750×g for five minutes at 4° C. 10 μL of each serum sample was loaded onto the center of the filter on the upper 96-well kit plate and dried in a stream of nitrogen. 20 μL of a 5% solution of phenyl-isothiocyanate was subsequently added for derivatization. The filter spots were then dried again using an evaporator. Extraction of the metabolites was then achieved by adding 300 μL methanol containing 5 mM ammonium acetate. The extracts were obtained by centrifugation into the lower 96-deep well plate. This was followed by a dilution step with MS running solvent from the AbsoluteIDQ® p180 Kit.

Mass spectrometric analysis was performed on the API4000 Qtrap® tandem mass spectrometer which was equipped with a solvent delivery system. The serum samples were delivered to the mass spectrometer by either a direct injection (DI) method or liquid chromatography method. The Biocrates MetIQ™ software, which is integral to the AbsolutelDQ® p180 Kit, was used to control the entire assay workflow, from sample registration to automated calculation of metabolite concentrations to the export of data into other data analysis programs. A targeted profiling scheme was used to quantitatively screen for known small molecule metabolites using multiple reaction monitoring, neutral loss, and precursor ion scans. Statistical analysis was performed using MetaboAnalyst (www.metaboanalyst.com) and ROCCET (www.roccet.ca).

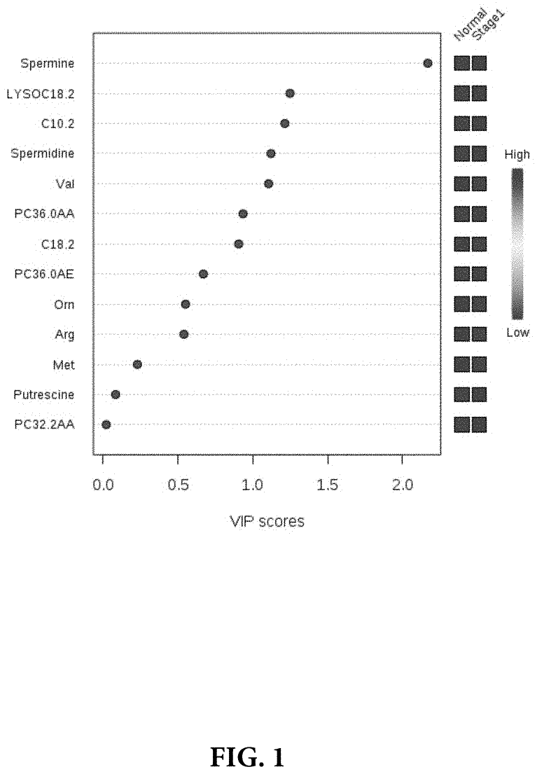

FIG. 1 is a variable importance in projection (VIP) plot showing the most discriminating serum metabolites in descending order of importance based on a partial least squares discriminant analysis (PLS-DA) showing separation between control patients and stage 1 lung cancer patients. A VIP score higher than 1.6 indicates highly significant metabolites. Table 2 shows the T-test statistics of discriminating serum metabolites from the VIP plot of FIG. 1.

TABLE 2
T-test statistics of serum metabolites for discriminating
stage 1 lung cancer.

| Metabolite | p. value | FDR |
|---|---|---|
| Spermine | 6.20E−06 | 8.06E−05 |
| Val | 0.0081354 | 0.044501 |
| LYSOC18.2 | 0.010578 | 0.044501 |
| C10.2 | 0.013692 | 0.044501 |
| PC36.0AA | 0.024106 | 0.062676 |
| C18.2 | 0.057366 | 0.12429 |
| PC36.0AE | 0.10719 | 0.19906 |
| Orn | 0.19421 | 0.29725 |
| Spermidine | 0.20579 | 0.29725 |
| Arg | 0.25549 | 0.33213 |
| Met | 0.58547 | 0.69191 |
| Putrescine | 0.85106 | 0.92198 |
| PC32.2AA | 0.96471 | 0.96471 |

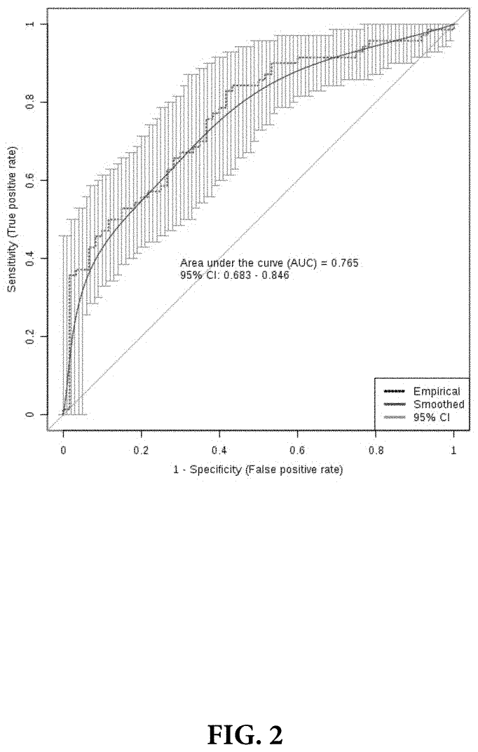

Using three serum metabolites identified from the VIP plot shown in FIG. 1, a logistic regression model was built to predict the probability of having stage 1 lung cancer with the following equation: logit(P)=log(P/(1−P))=0.217−1.241×Spermine−0.598×LYSOC18.22−0.817×C10.2. FIG. 2 shows an area under the receiver operating characteristic curve (AUROC) generated by the equation.

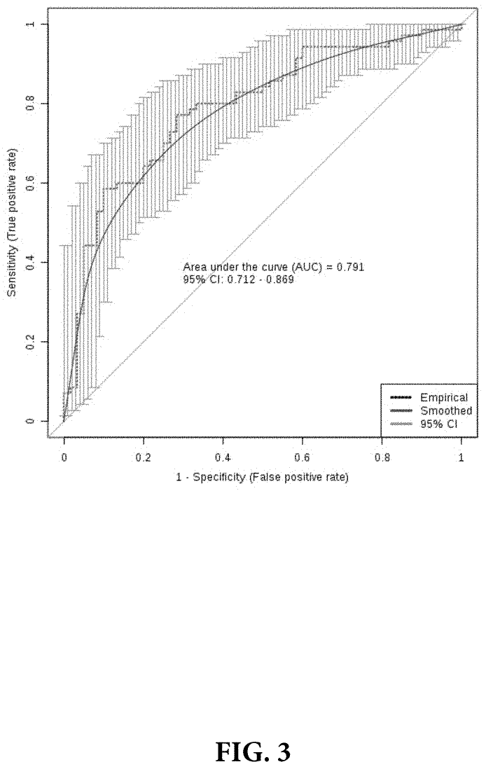

Another logistic regression model was built to predict the probability of having stage 1 lung cancer using six serum metabolites identified from the VIP plot shown in FIG. 1 with the following equation: logit(P)=log(P/(1−P))=0.243−1.131×Spermine −0.62×LYSOC18.2−0.92×C10.2+0.642×Val−0.825×PC36.0AA+0.573×C18.2. FIG. 3 shows an AUROC curve generated by the equation.

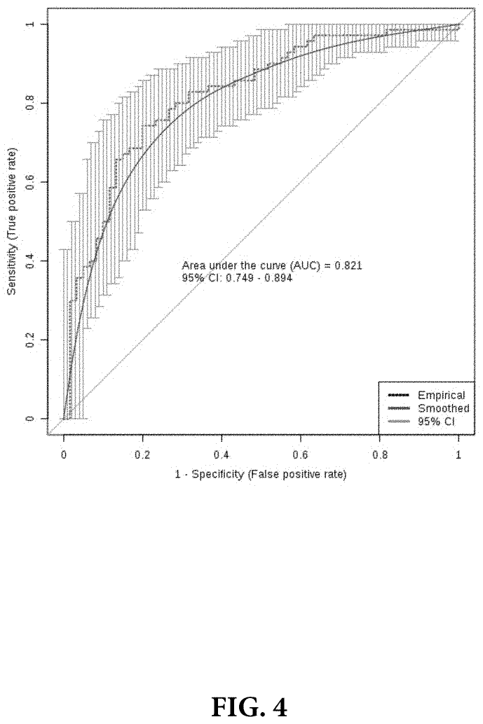

FIG. 4 shows an AUROC curve generated by a logistic regression model for predicting the probability of having stage 1 lung cancer using three serum metabolites identified from the VIP plot shown in FIG. 1 and taking into account smoking status with the following equation: logit(P)=log(P/(1−P))=0.207+0.32×Smoking Status−1.18×Spermine−0.472×LYSOC18.2−0.724×C10.2.

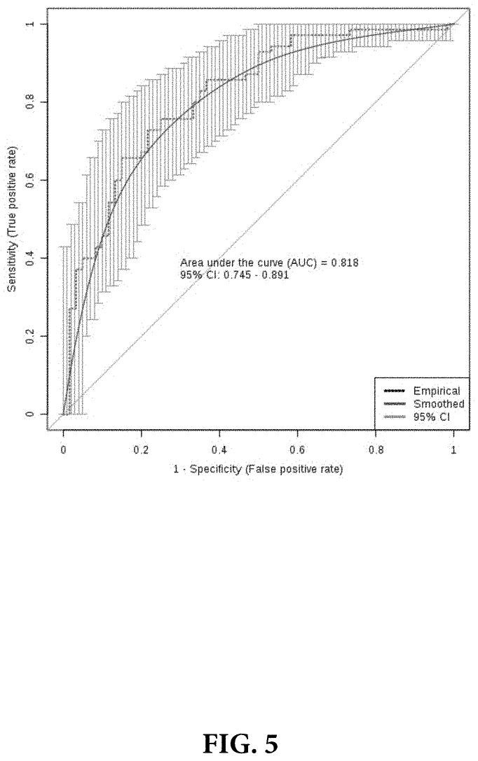

FIG. 5 shows an AUROC curve generated by a logistic regression model for predicting the probability of having stage 1 lung cancer using three serum metabolites and taking into account smoking status and body mass index (BMI) with the following equation: logit(P)=log(P/(1−P))=0.215−1.279×Spermine−0.42×LYSOC18.2−0.748×C10.2+0.507×BMI+0.294×Smoking Status.

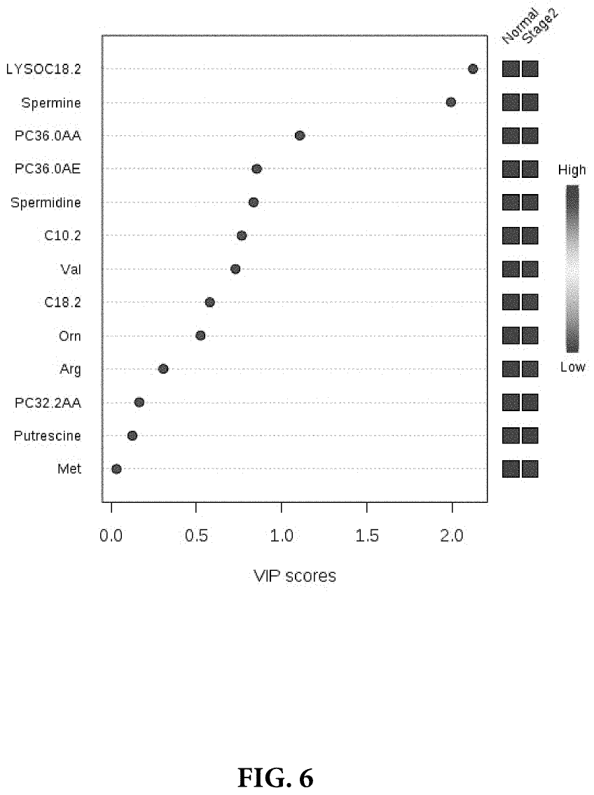

FIG. 6 is a VIP plot showing the most discriminating serum metabolites in descending order of importance based on a PLS-DA analysis showing separation between control patients and stage 2 lung cancer patients. A VIP score higher than 1.6 indicates highly significant metabolites. Table 3 shows the T-test statistics of discriminating serum metabolites from the VIP plot of FIG. 6.

TABLE 3
T-test statistics of serum metabolites for discriminating
stage 2 lung cancer.

| Metabolite | p. value | FDR |
|---|---|---|
| Spermine | 3.42E−09 | 4.45E−08 |
| LYSOC18.2 | 7.60E−08 | 4.94E−07 |
| PC36.0AA | 0.00012008 | 0.00052034 |
| PC36.0AE | 0.0032963 | 0.010713 |
| Val | 0.014762 | 0.03838 |
| C10.2 | 0.029034 | 0.062906 |
| Orn | 0.071816 | 0.13337 |
| C18.2 | 0.10904 | 0.17719 |
| Spermidine | 0.15233 | 0.22003 |
| Arg | 0.38045 | 0.49458 |
| PC32.2AA | 0.611 | 0.72209 |
| Putrescine | 0.70261 | 0.76116 |
| Met | 0.91798 | 0.91798 |

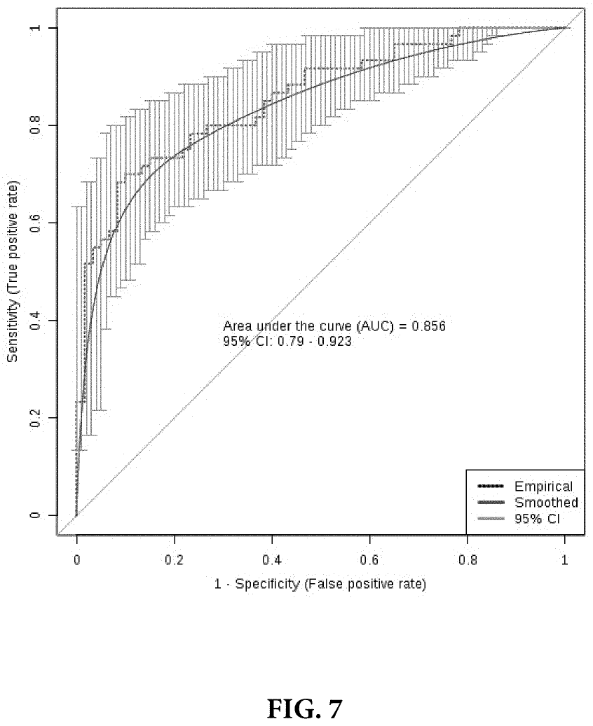

Using two serum metabolites identified from the VIP plot shown in FIG. 6, a logistic regression model was built to predict the probability of having stage 2 lung cancer with the following equation: logit(P)=0.088−1.728×Spermine−1.484×LYSOC18.2. FIG. 7 shows an AUROC curve generated by the equation.

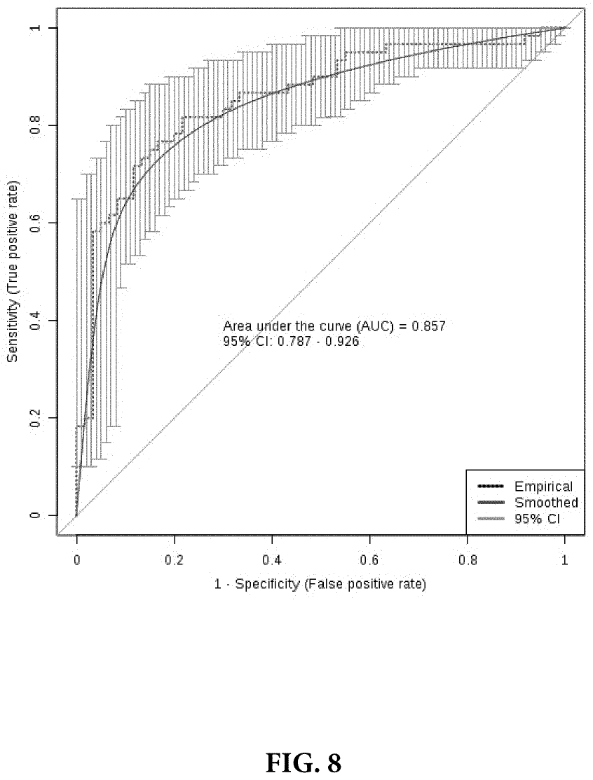

Another logistic regression model was built using seven serum metabolites identified from the VIP plot shown in FIG. 6 to predict the probability of having stage 2 lung cancer with the following equation: logit(P)=log(P/(1−P))=0.172−1.647×Spermine−1.346×LYSOC18.2−1.521×PC36.0AA+0.215×PC36.0AE+0.563×Val −0.358×C10.2+0.757×Orn. FIG. 8 shows an AUROC curve generated by the equation.

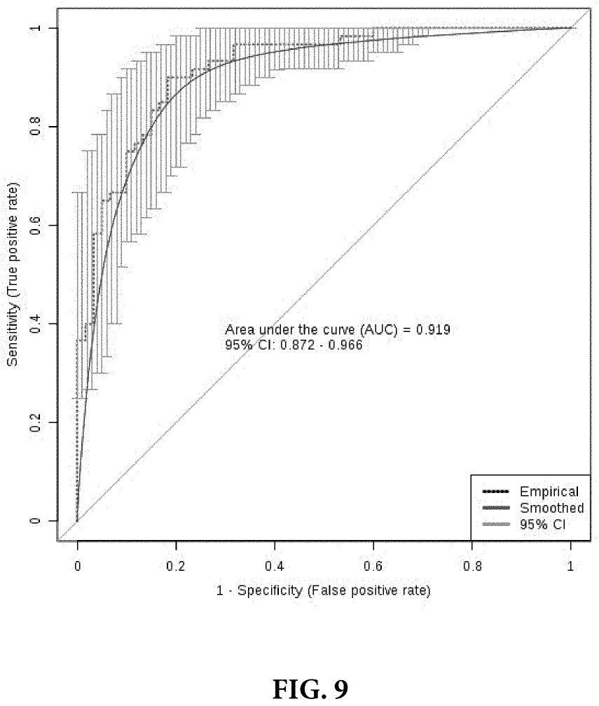

FIG. 9 shows an AUROC curve generated by a logistic regression model for predicting the probability of having stage 2 lung cancer using three serum metabolites identified from the VIP plot shown in FIG. 6 and taking into account smoking status with the following equation: logit(P)=log(P/(1−P))=−0.107−1.903×Spermine+0.632×Smoking Status−0.882×LYSOC18.2−1.549×PC36.0AA.

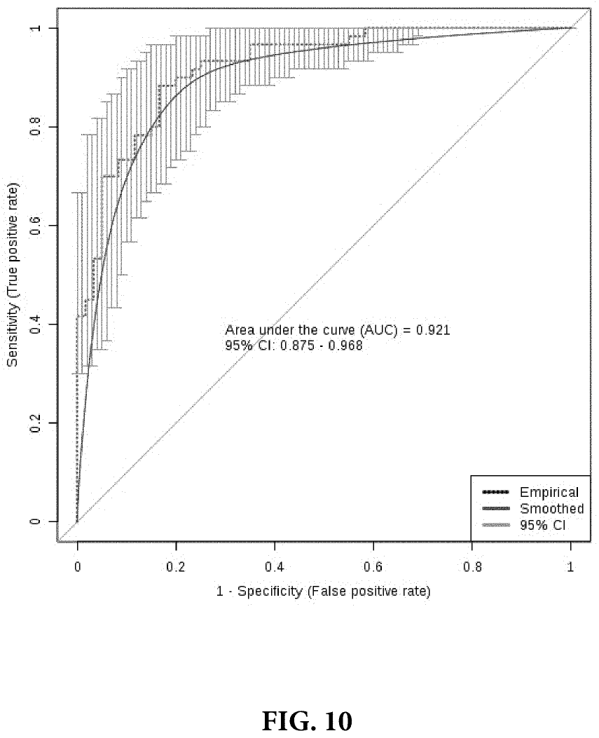

FIG. 10 shows an AUROC curve generated by a logistic regression model for predicting the probability of having stage 2 lung cancer using three serum metabolites identified from the VIP plot shown in FIG. 6 and taking into account smoking status and BMI with the following equation: logit(P)=log(P/(1−P))=−0.132−0.917×LYSOC18.2−1.91×Spermine+0.661×Smoking Status−1.518×PC36.0AA−0.419×BMI.

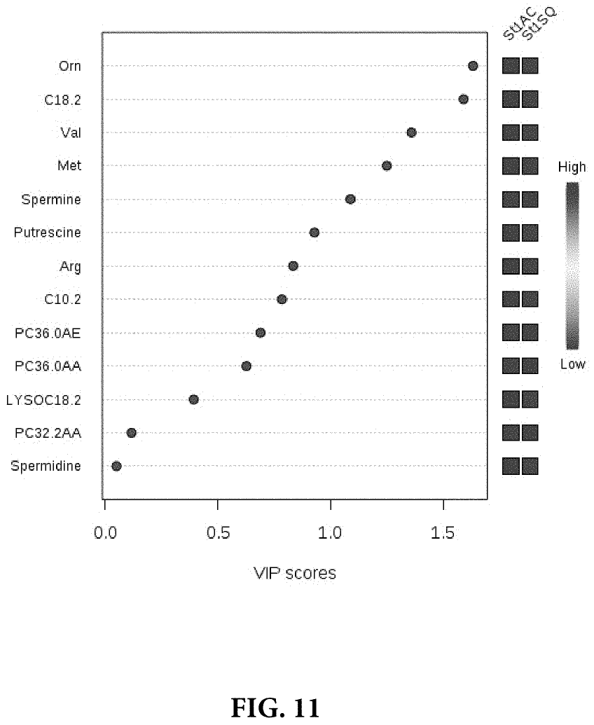

FIG. 11 is a VIP plot showing the most discriminating serum metabolites in descending order of importance based on a PLS-DA analysis showing separation between stage 1 adenocarcinoma lung cancer patients and stage 1 squamous lung cancer patients. A

VIP score higher than 1.6 indicates highly significant metabolites. Table 4 shows the T-test statistics of discriminating serum metabolites from the VIP analysis of FIG. 11.

TABLE 4
T-test statistics of serum metabolites for discriminating stage 1
adenocarcinoma lung cancer and stage 1 squamous lung cancer.

| Metabolite | p. value | FDR |
|---|---|---|
| Orn | 0.0096012 | 0.11067 |
| Val | 0.02525 | 0.11067 |
| C18.2 | 0.02554 | 0.11067 |
| Met | 0.035935 | 0.11679 |
| Spermine | 0.12507 | 0.32518 |
| Putrescine | 0.17277 | 0.37434 |
| Arg | 0.24145 | 0.39447 |
| C10.2 | 0.24696 | 0.39447 |
| PC36.0AE | 0.27309 | 0.39447 |
| PC36.0AA | 0.32467 | 0.42207 |
| LYSOC18.2 | 0.57942 | 0.68477 |
| PC32.2AA | 0.87243 | 0.94513 |
| Spermidine | 0.971 | 0.971 |

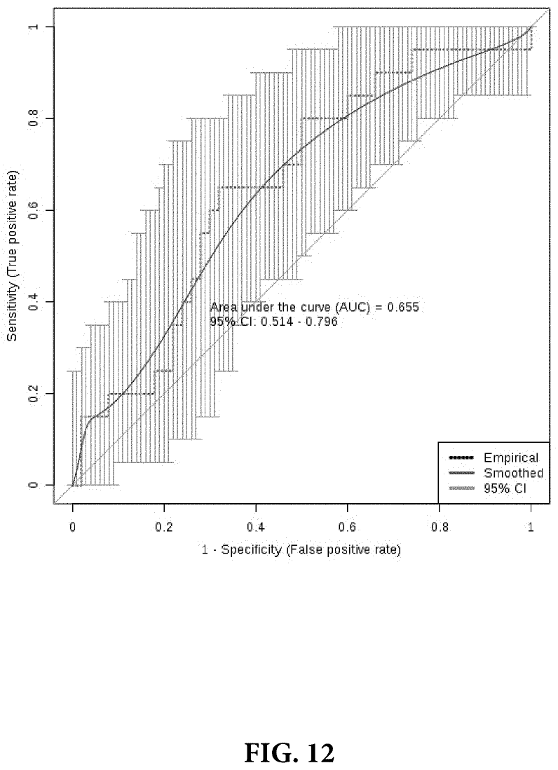

Using four serum metabolites identified from the VIP plot shown in FIG. 11, a logistic regression model was built to predict the probability of having stage 1 adenocarcinoma lung cancer versus stage 1 squamous lung cancer with the following equation: logit(P)=logit(P)=log(P/(1−P))=−1.074+0.588×Orn+0.614×C18.2+0.547×Val+0.141×Met. FIG. 12 shows an AUROC curve generated by the equation.

Another logistic regression model was built using four serum metabolites identified from the VIP plot shown in FIG. 11 and taking into account smoking history to predict the probability of having stage 1 adenocarcinoma lung cancer versus stage 1 squamous lung cancer with the following equation: logit(P)=log(P/(1−P))=0.172−1.647×Spermine−1.346×LYSOC18.2−1.521×PC36.0AA+0.215×PC36.0AE+0.563×Val−0.358×C10.2+0.757×Orn. FIG. 8 shows an AUROC curve generated by the equation.

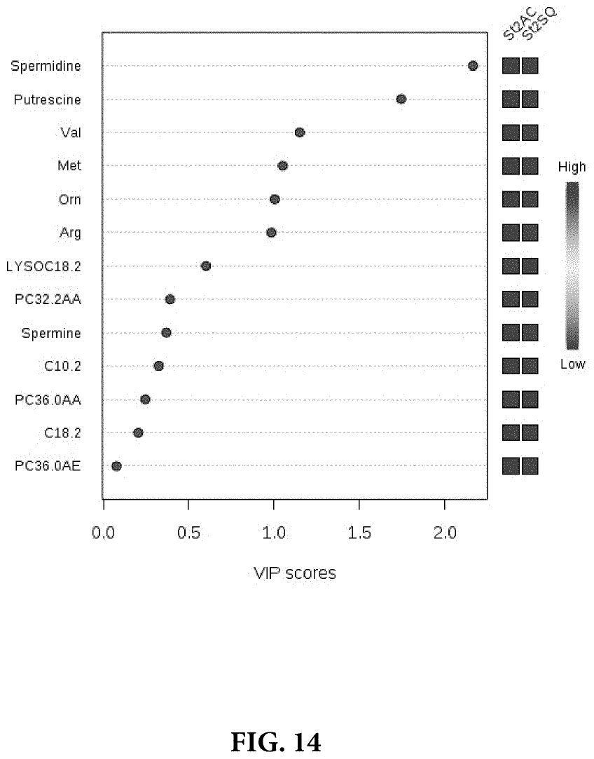

FIG. 14 is a VIP plot showing the most discriminating serum metabolites in descending order of importance based on a PLS-DA analysis showing separation between stage 2 adenocarcinoma lung cancer patients and stage 2 squamous lung cancer patients. A VIP score higher than 1.6 indicates highly significant metabolites. Table 5 shows the T-test statistics of discriminating serum metabolites from the VIP analysis of FIG. 14.

TABLE 5
T-test statistics of serum metabolites for differentiating
between stage 2 adenocarcinoma lung cancer and
stage 2 squamous lung cancer.

| Metabolite | p. value | FDR |
|---|---|---|
| Spermine | 3.42E−09 | 4.45E−08 |
| LYSOC18.2 | 7.60E−08 | 4.94E−07 |
| PC36.0AA | 0.00012008 | 0.00052034 |
| PC36.0AE | 0.0032963 | 0.010713 |
| Val | 0.014762 | 0.03838 |
| C10.2 | 0.029034 | 0.062906 |
| Orn | 0.071816 | 0.13337 |
| C18.2 | 0.10904 | 0.17719 |
| Spermidine | 0.15233 | 0.22003 |
| Arg | 0.38045 | 0.49458 |
| PC32.2AA | 0.611 | 0.72209 |
| Putrescine | 0.70261 | 0.76116 |
| Met | 0.91798 | 0.91798 |

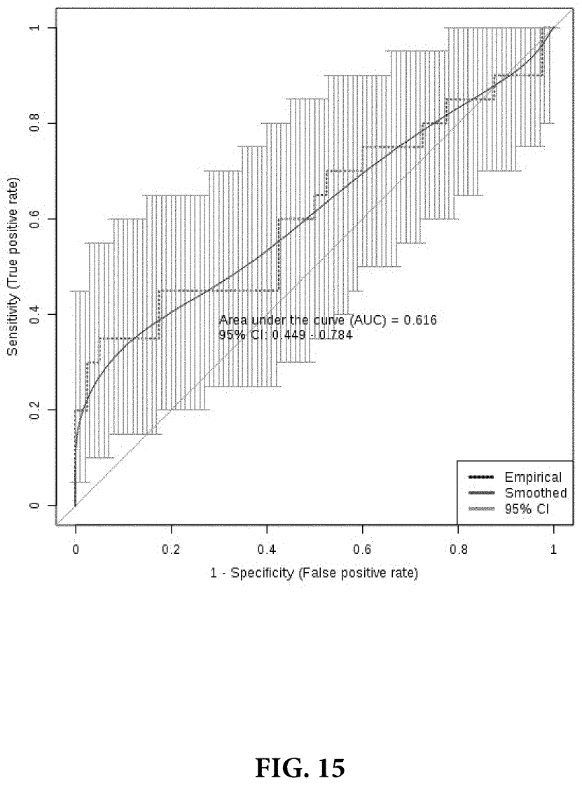

Using four serum metabolites identified from the VIP plot shown in FIG. 14, a logistic regression model was built to predict the probability of having stage 2 adenocarcinoma lung cancer versus stage 2 squamous lung cancer with the following equation: log(P/(1−P))=−0.825+0.466×Spermidine+0.662×Putrescine+0.762×Val−0.406×Met. FIG. 15 shows an AUROC curve generated by the equation.

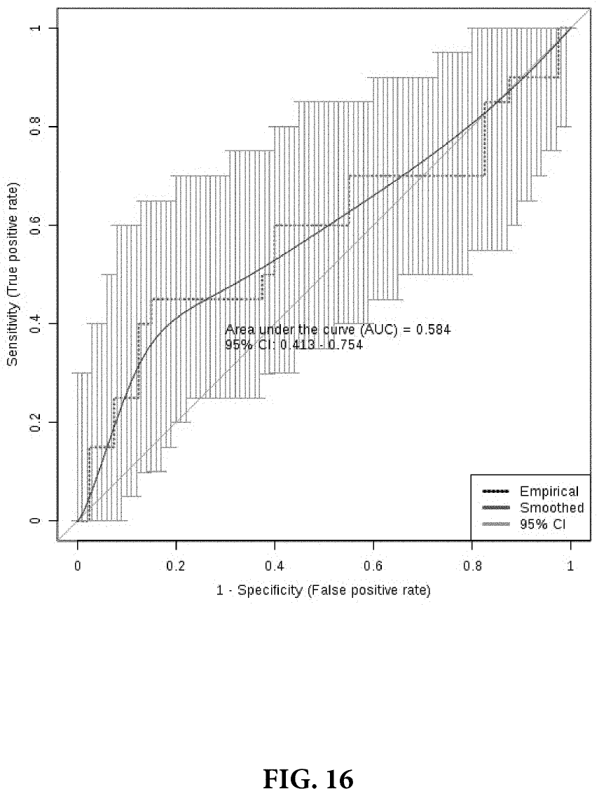

Another logistic regression model was built using the seven most important serum metabolites to predict the probability of having stage 2 adenocarcinoma lung cancer versus stage 2 squamous lung cancer with the following equation: logit(P)=log(P/(1−P))=−0.95+0.872×Spermidine−0.327×LYSOC18.2−2.125×PC36.0AA+1.63×PC36.0AE+1.068×Val+0.445×C10.2−0.105×Orn. FIG. 16 shows an AUROC curve generated by the equation.

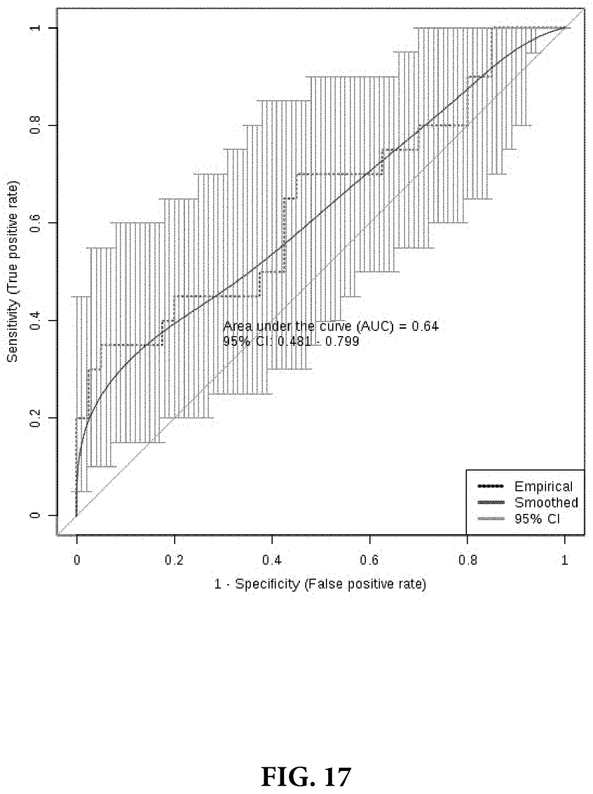

FIG. 17 shows an AUROC curve generated by a logistic regression model for predicting the probability of having stage 2 adenocarcinoma lung cancer versus stage 2 squamous lung cancer, using the four most important serum metabolites and taking into account smoking status with the following equation: logit(P)=log(P/(1−P))=−0.941+0.361×Spermidine+0.595×Putrescine+0.787×Val−0.358×Met+0.416×Smoking Status.

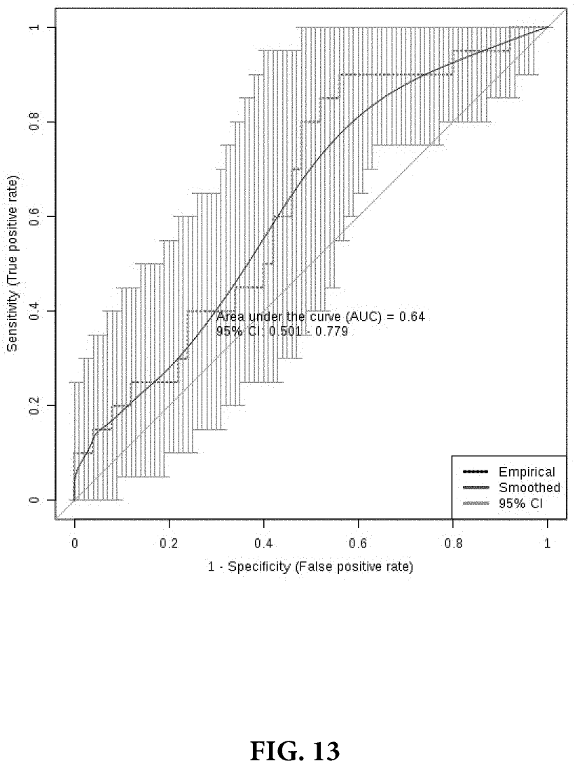

The results described above and shown in FIGS. 1 to 17 indicate that 13 metabolites have been identified as putative biomarkers for lung cancer, namely, arginine, C18.2 decadienylcarnitine (C10:2), LYSOC18.2, methionine, ornithine, PC32:2AA, PC36.0AA, PC36.0AE, putrescine, spermidine, spermine, and valine. These metabolites may be used in a biomarker panel to detect lung cancer.

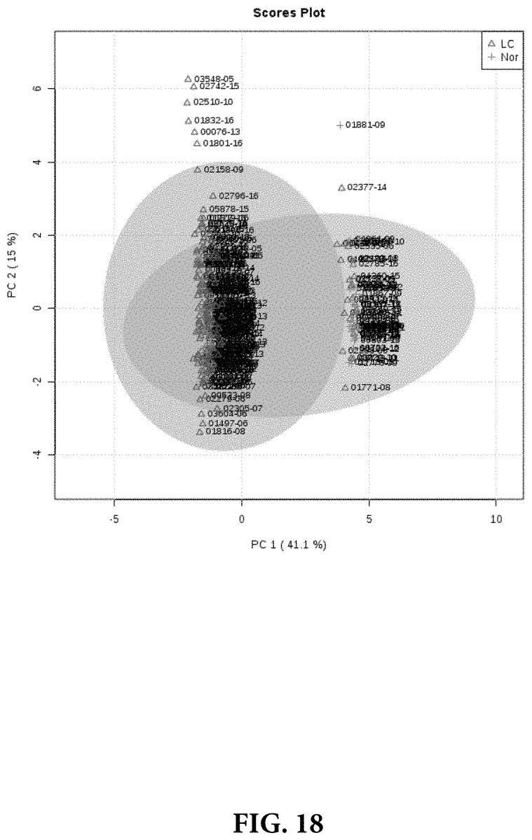

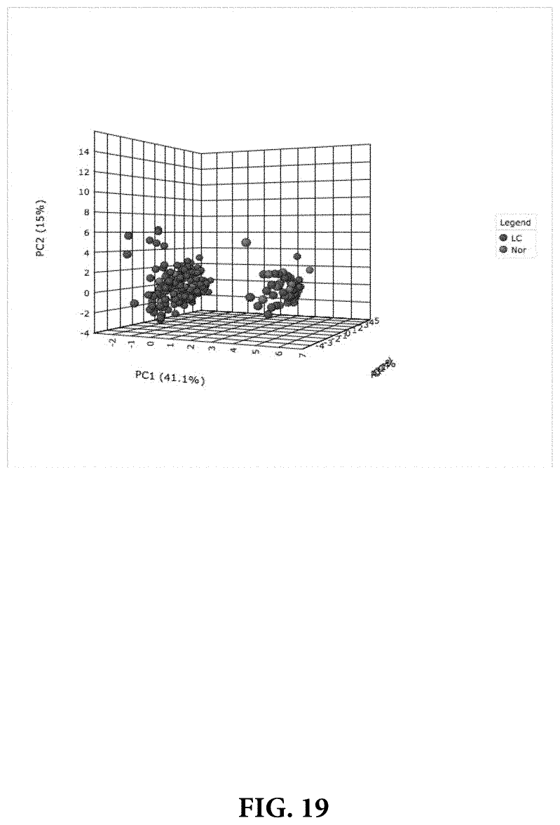

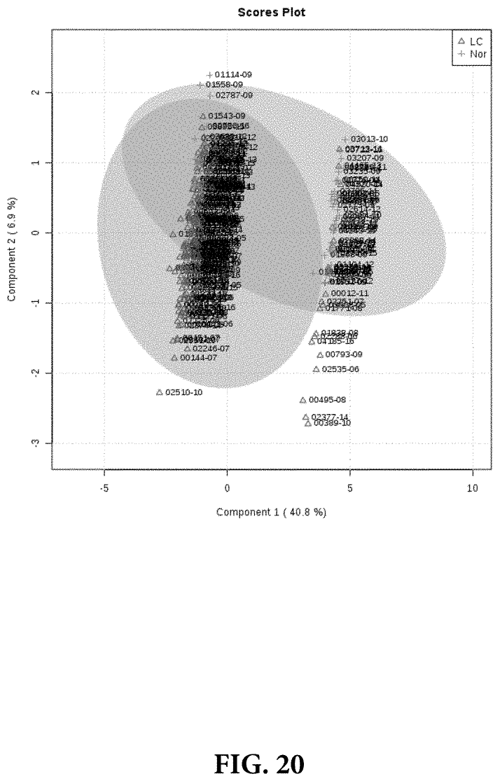

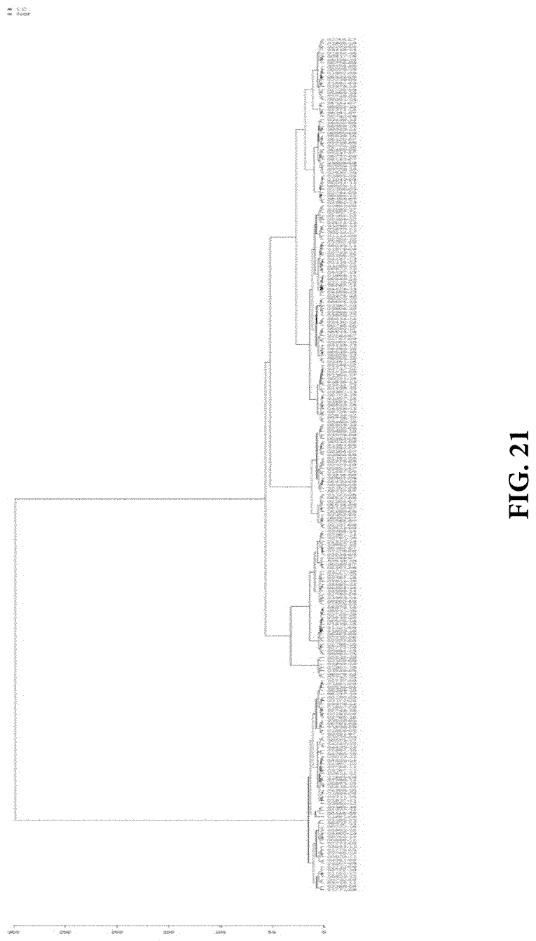

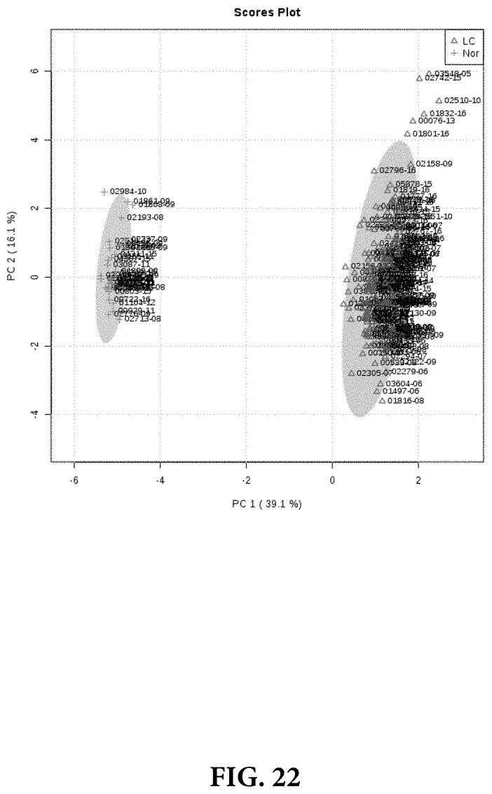

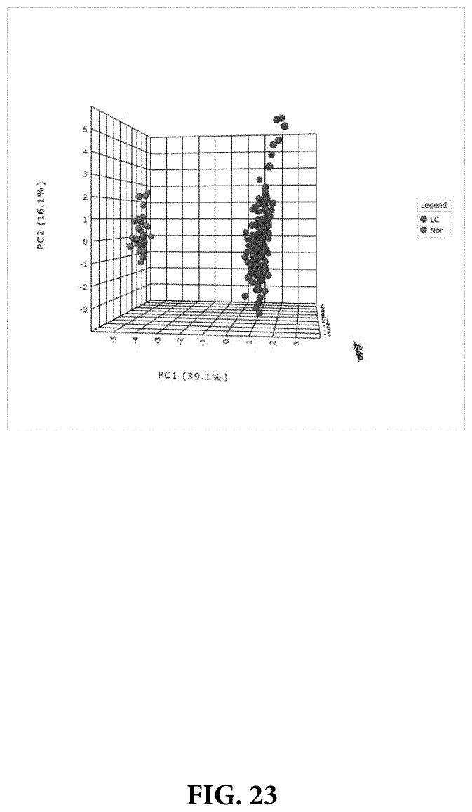

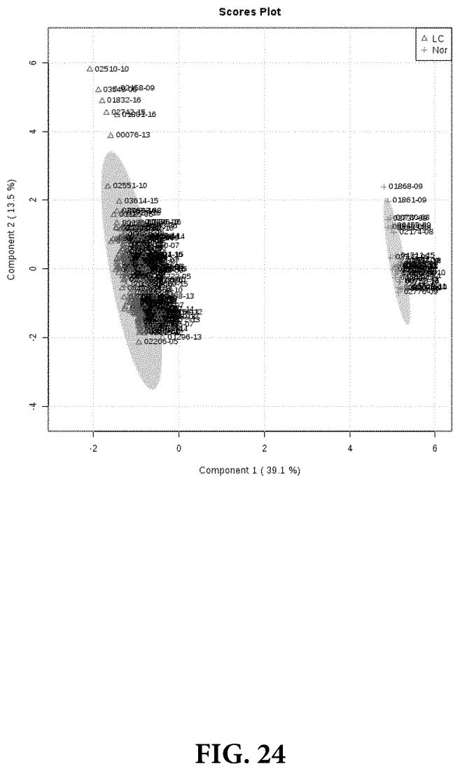

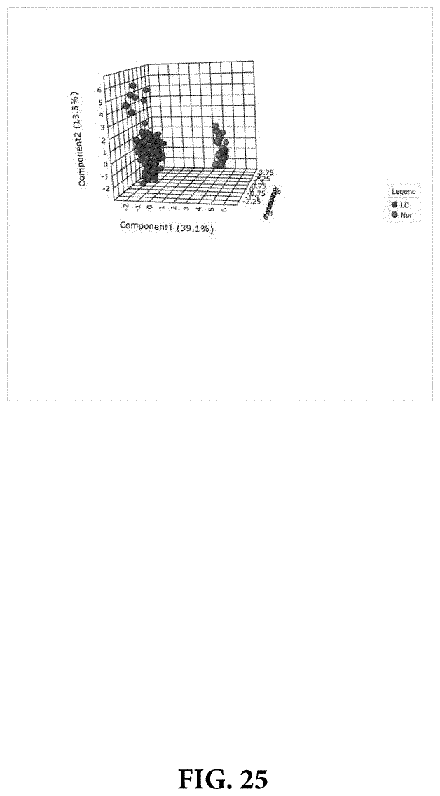

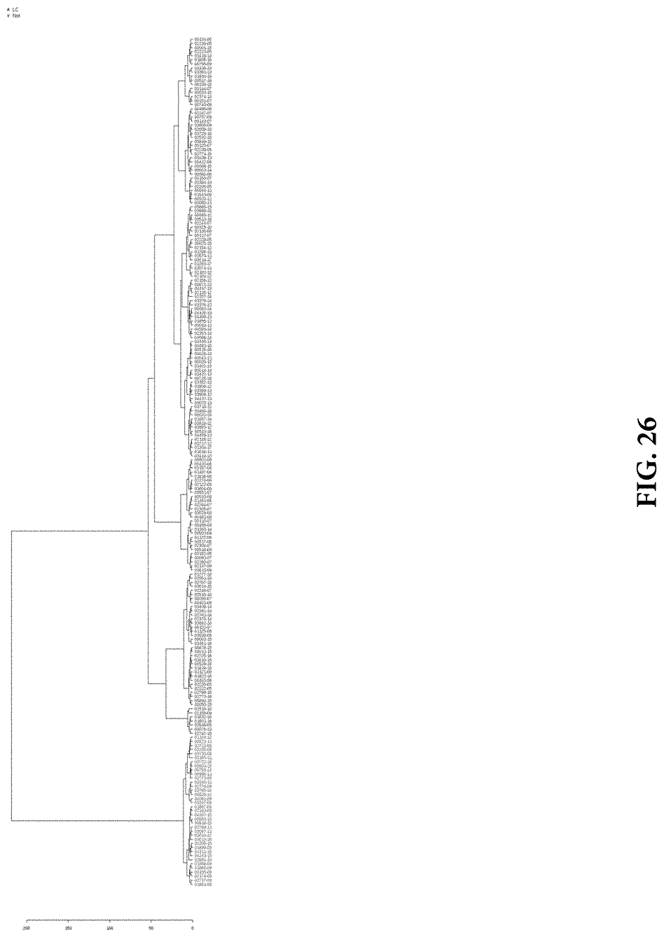

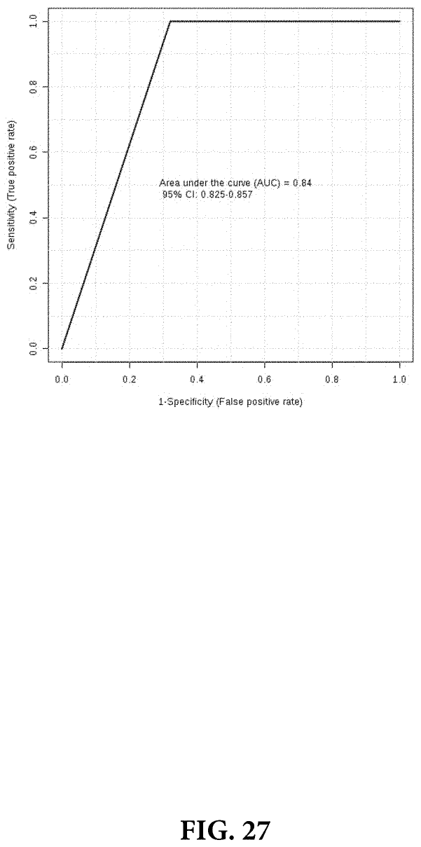

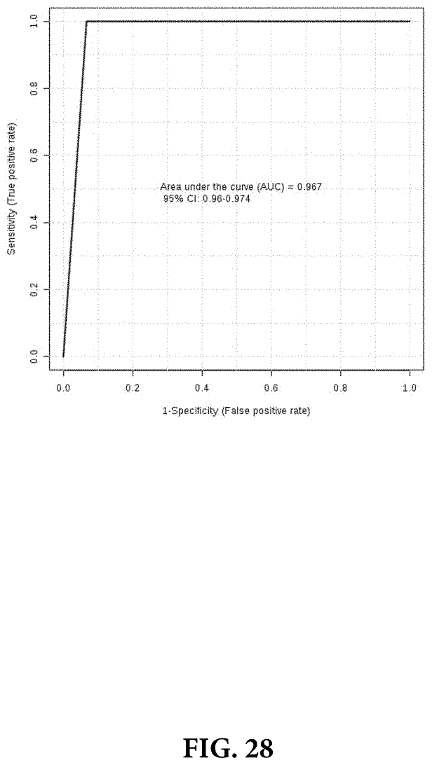

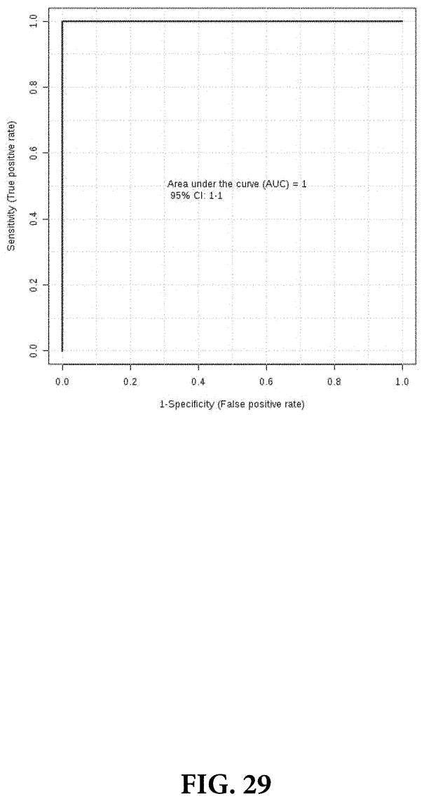

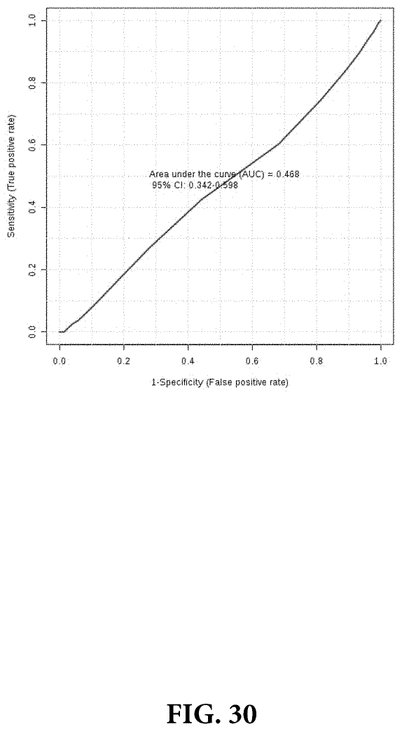

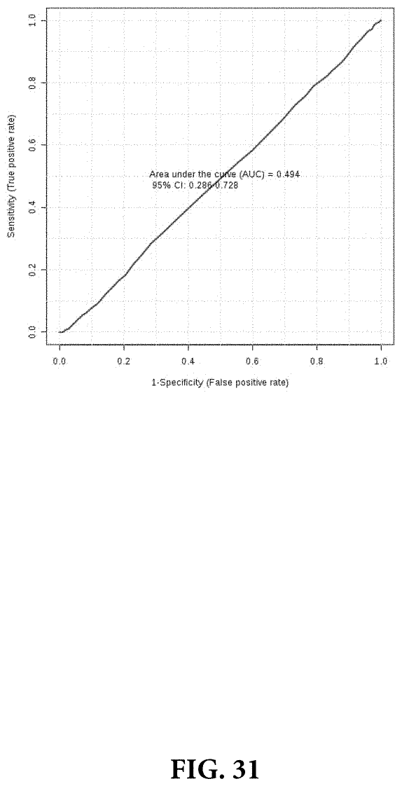

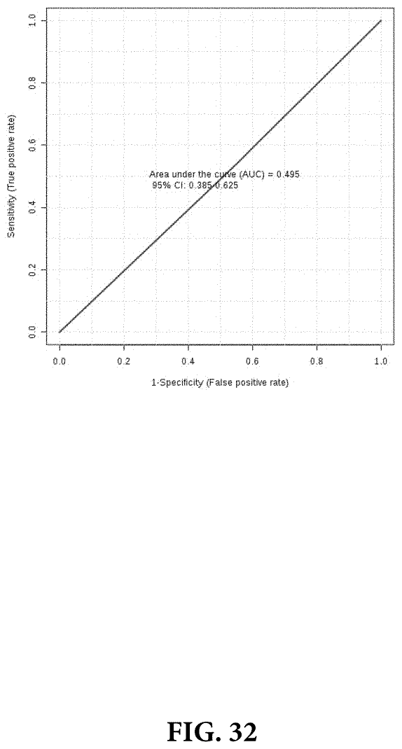

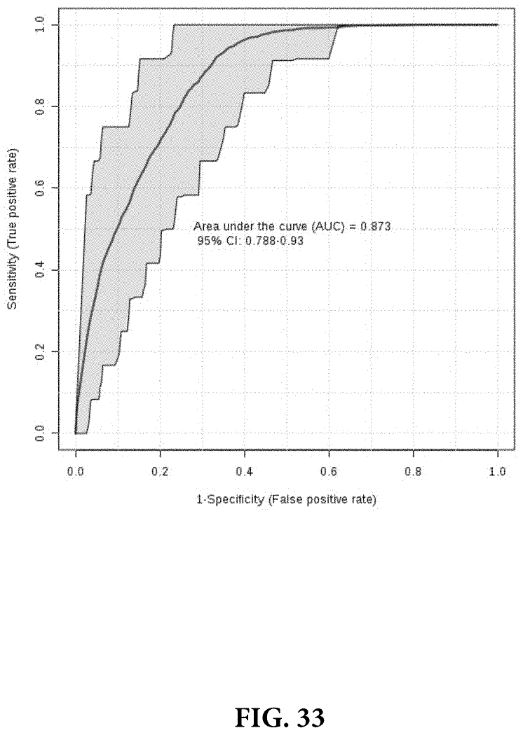

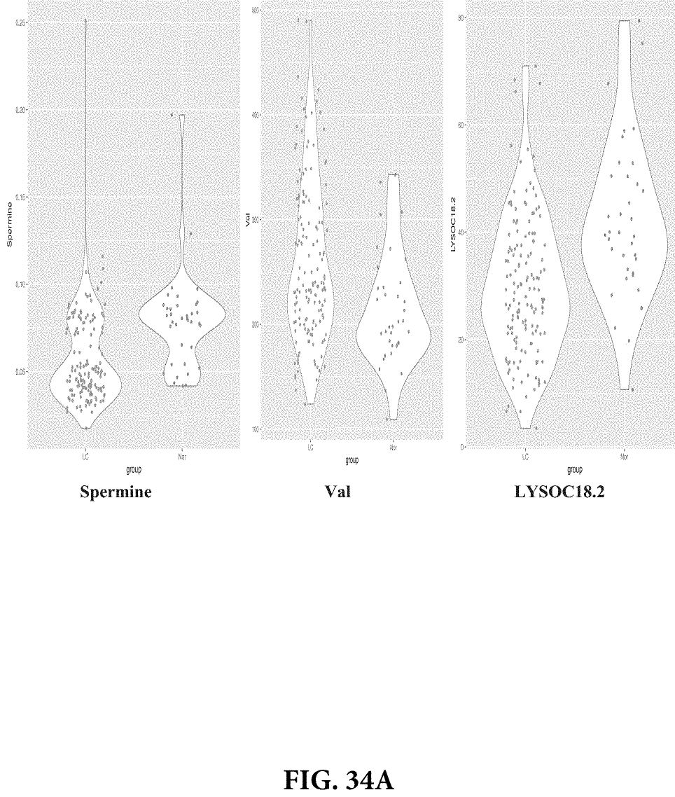

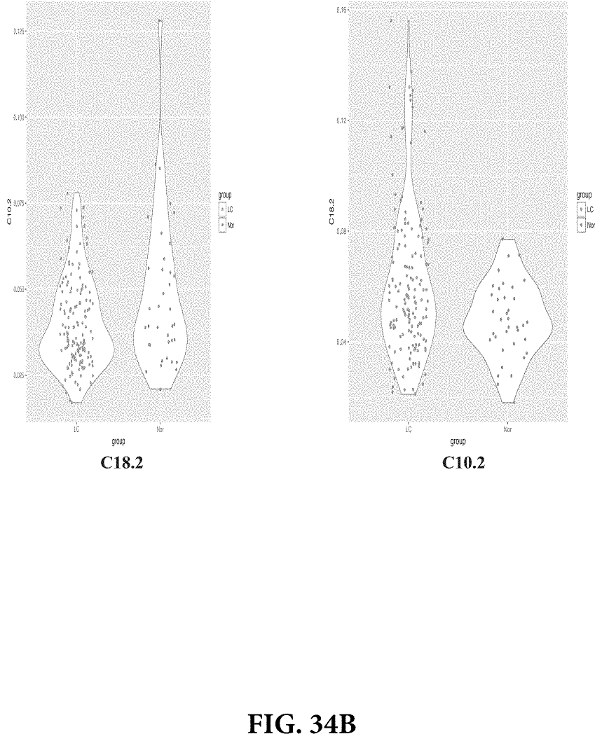

FIGS. 18-26 show the data-preprocessing for Groups 1-9 patients vs. control patients. FIGS. 27-32 show the clinical factor contributions for the total lung cancer patients (Groups 1-6) vs. control patients. Smoking seems to be the best clinical variable, and specifically consumption. FIGS. 33-34 analyze metabolites for total lung cancer patients (Groups 1-6) vs. control patients. FIG. 33 demonstrates the robustness of the assay; an AUC score of 0.873 is achieved using only metabolites. But including both the metabolites and smoking (cigarette consumption) increases the AUC score to 0.967.

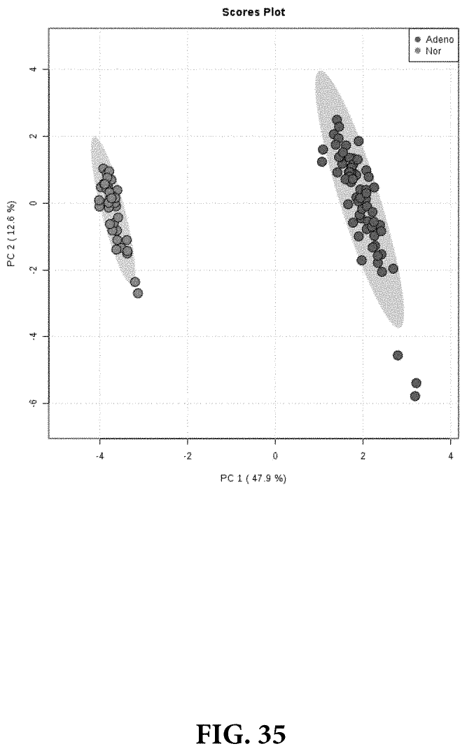

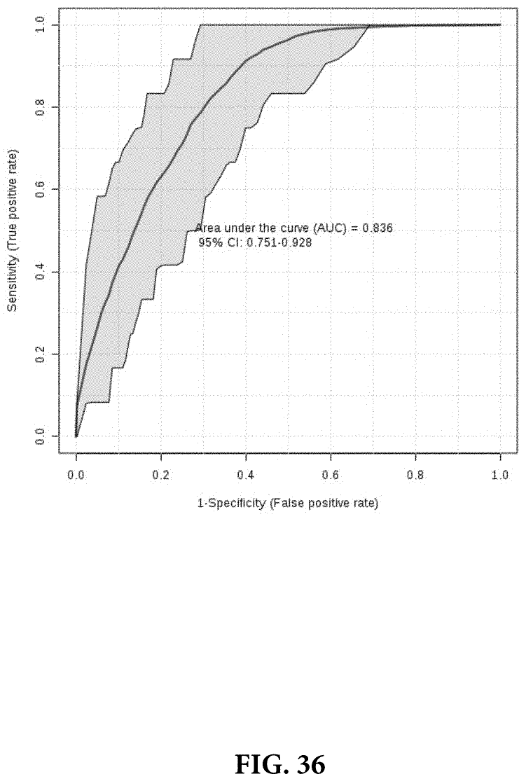

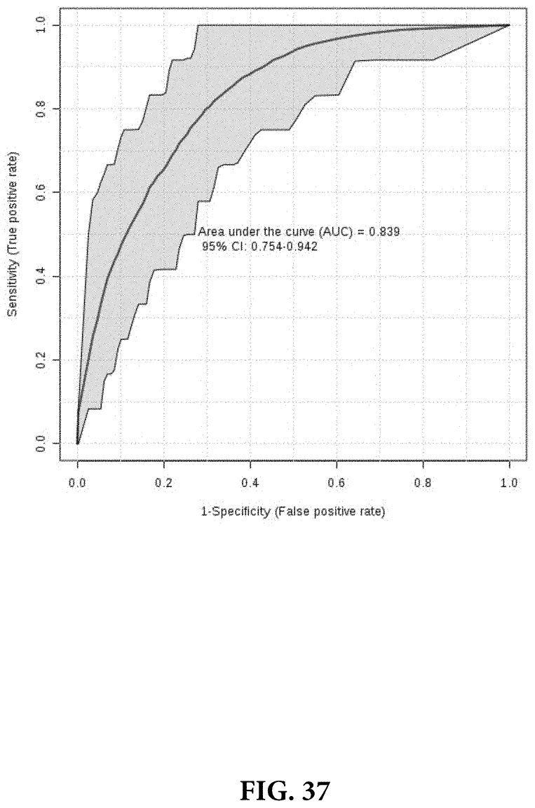

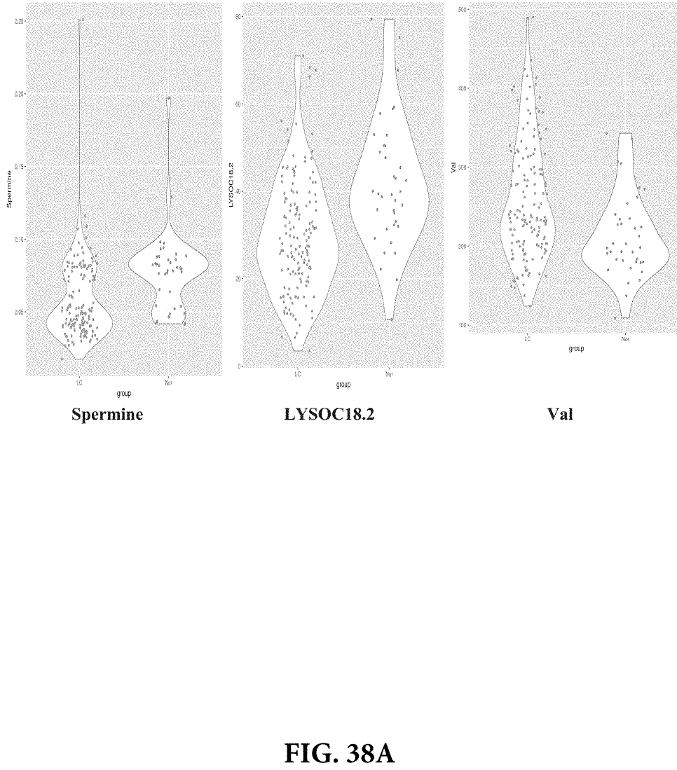

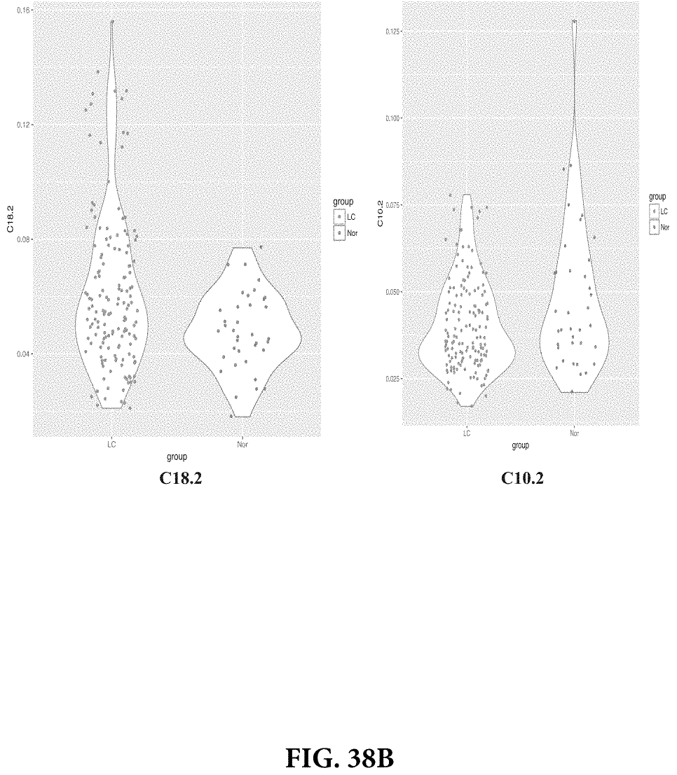

FIGS. 35-38 analyze metabolites for diagnosing stage 1 and 2 adenocarcinoma lung cancer patients.

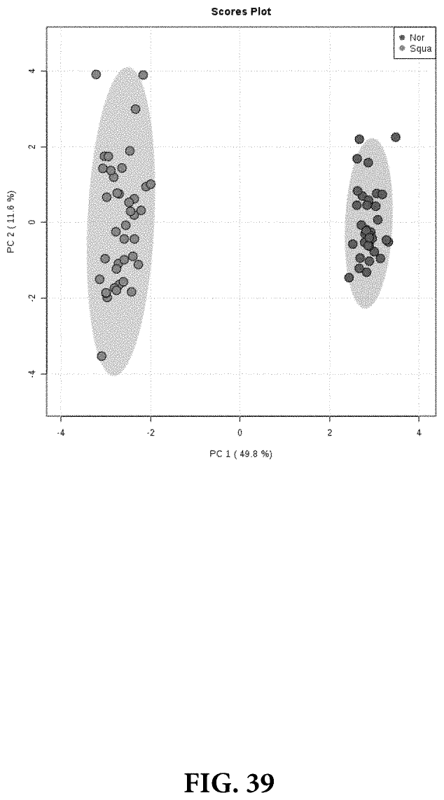

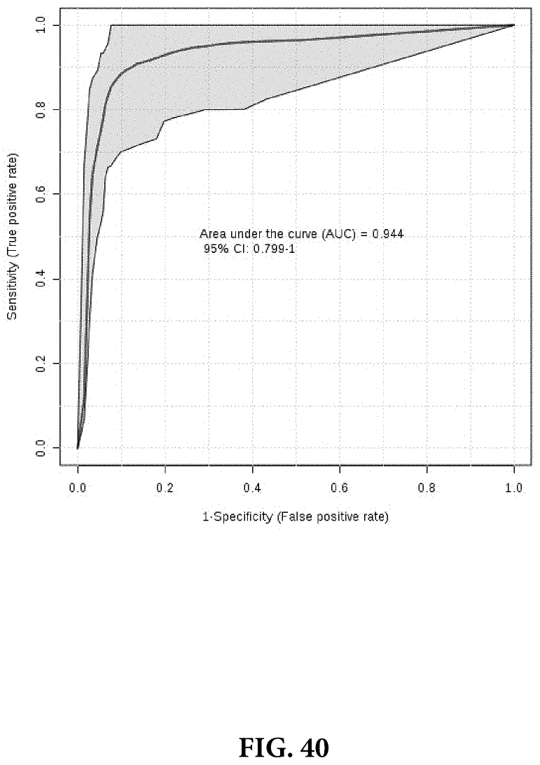

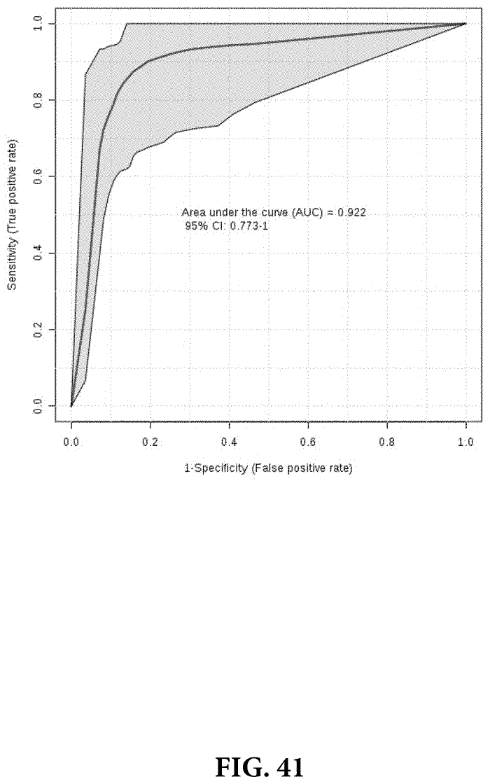

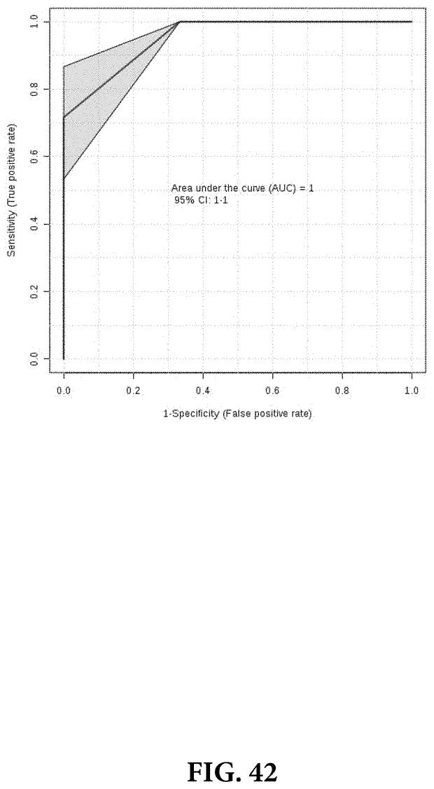

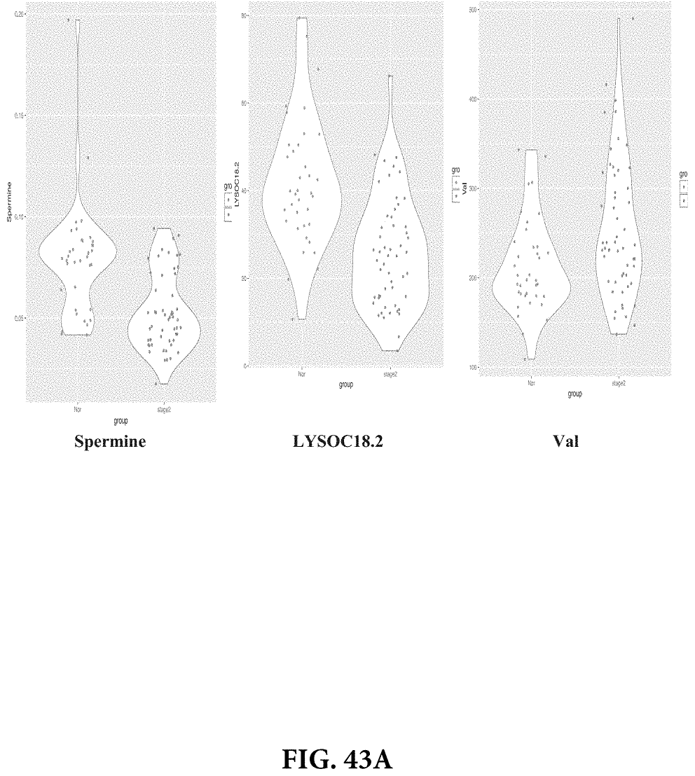

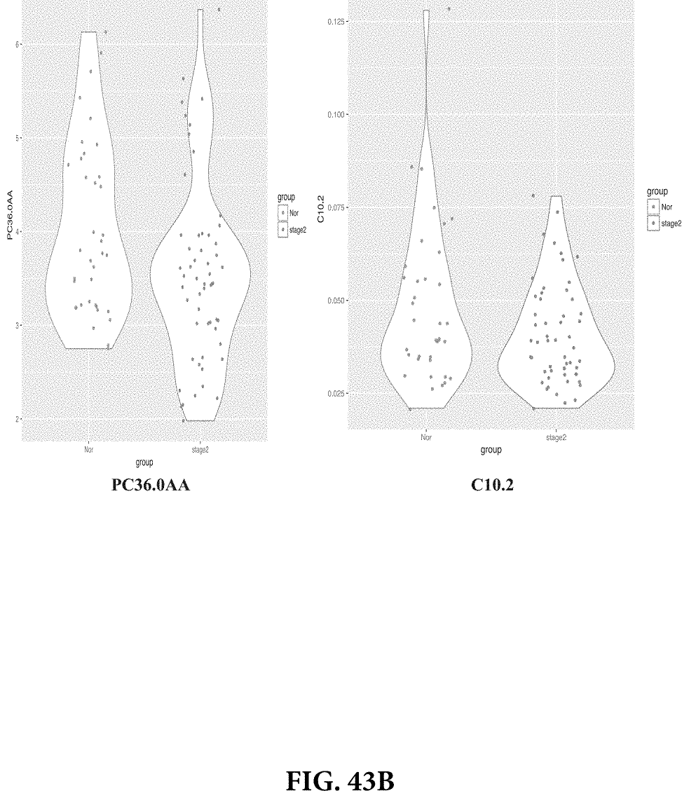

FIGS. 39-43 analyze metabolites and clinical factor contributions for diagnosing combined stage 1 and 2 squamous lung cancer patients. FIG. 41 shows that using metabolites and BMI for diagnosing combined stage 1 and 2 squamous lung cancer patients achieves an AUC score of 0.922. With smoking added, the AUC score is well over 0.97.

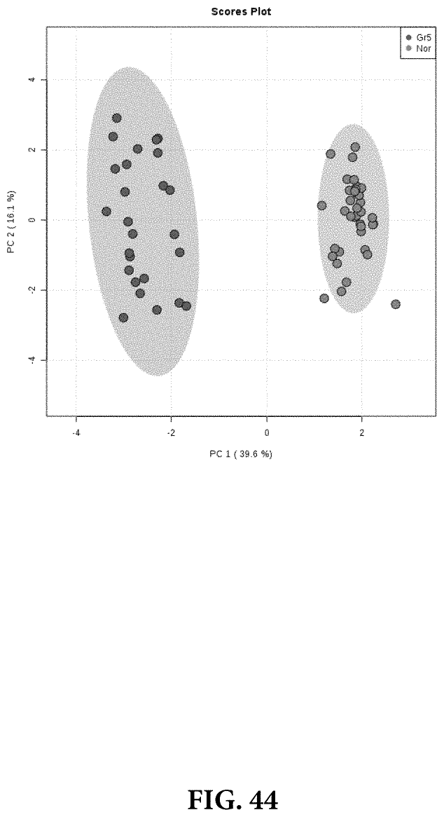

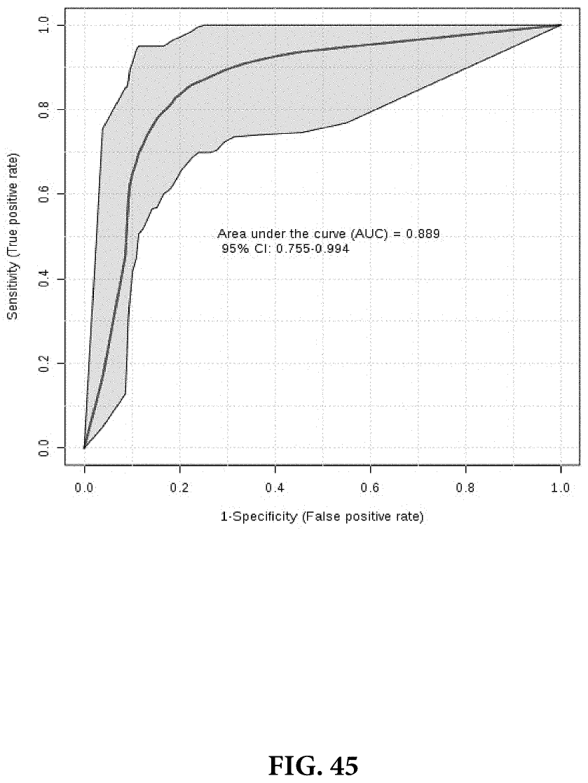

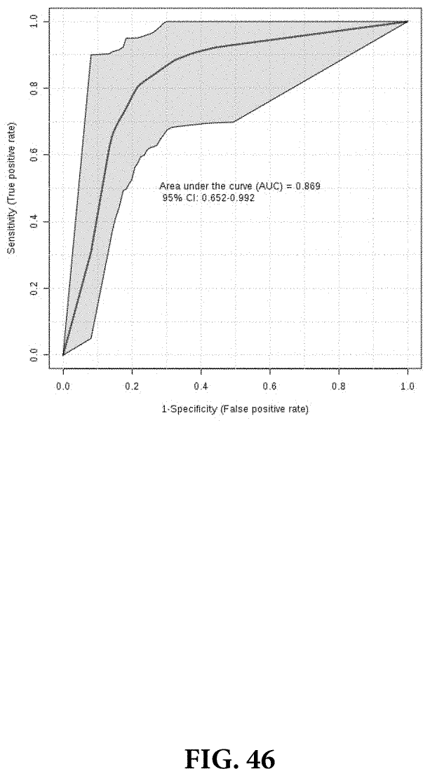

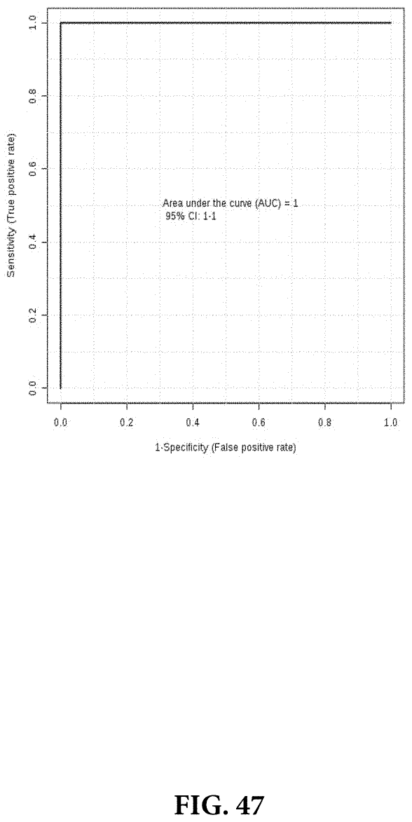

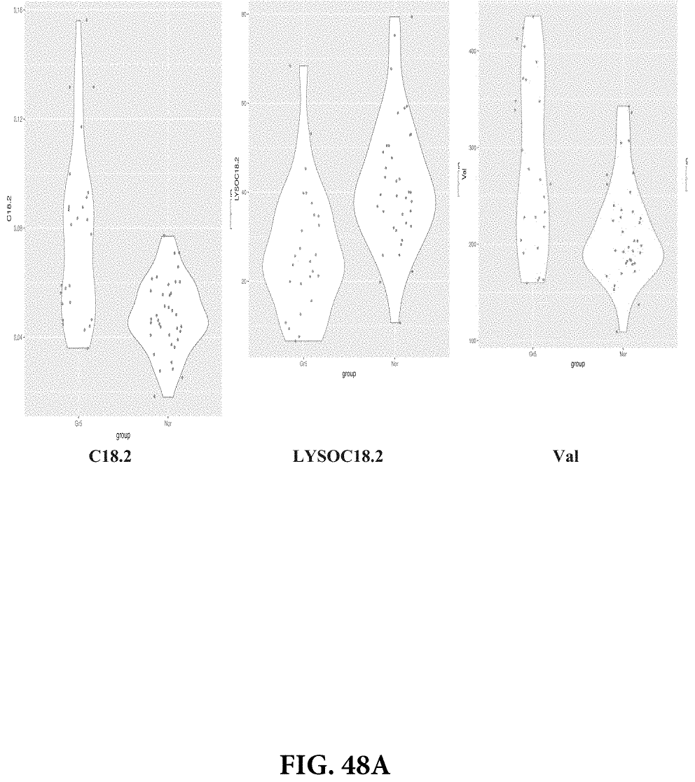

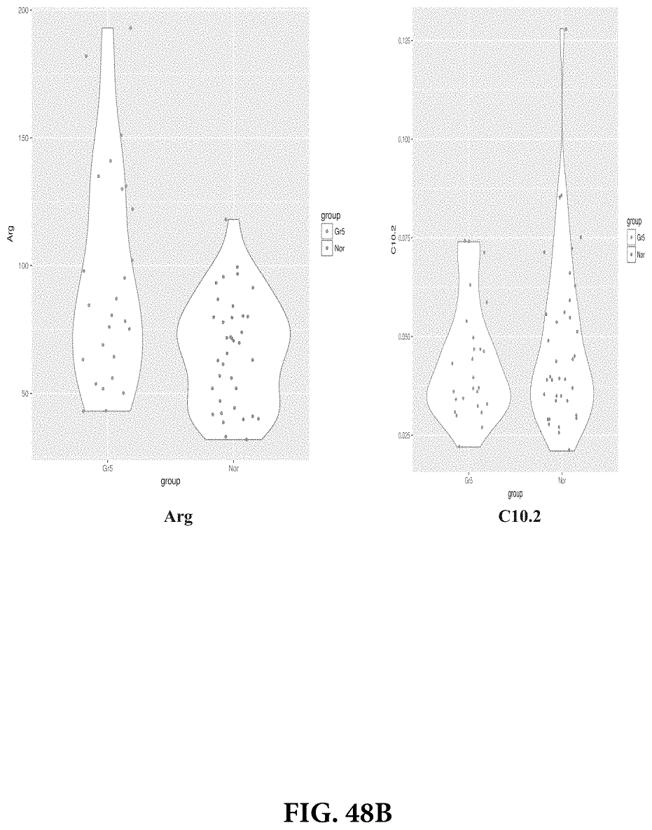

FIGS. 44-48 analyze metabolites and clinical factor contributions for diagnosing stage 3b/4 NSCLC lung cancer patients (Group 5).

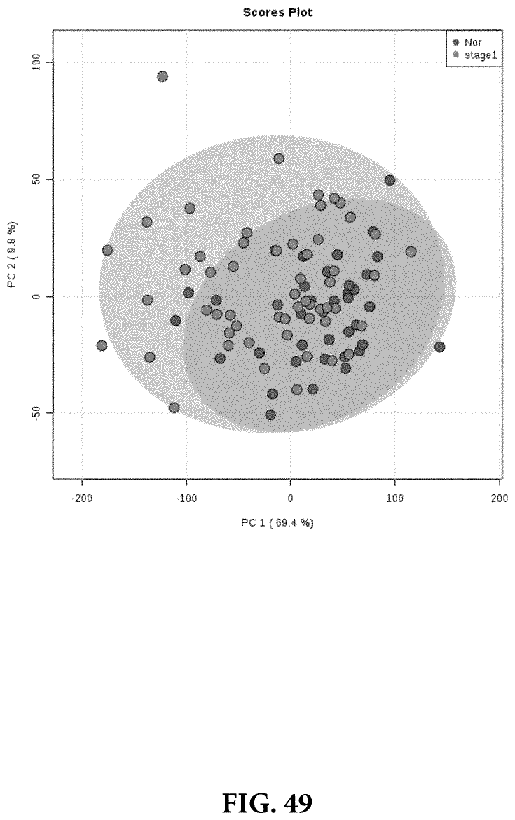

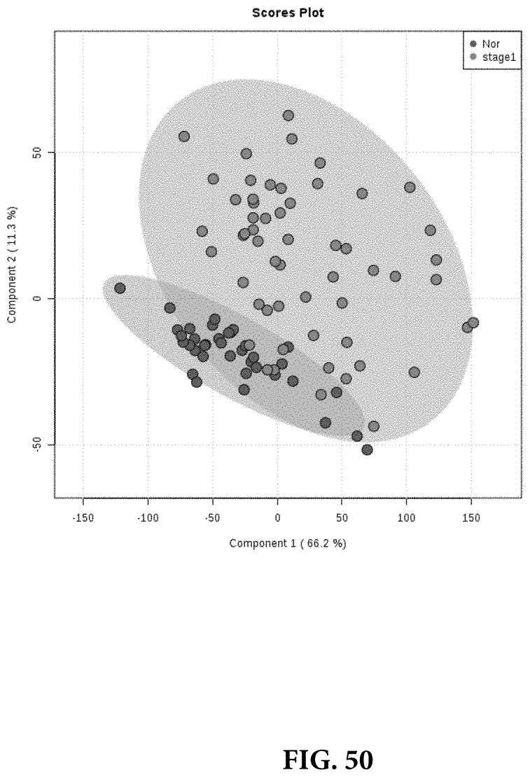

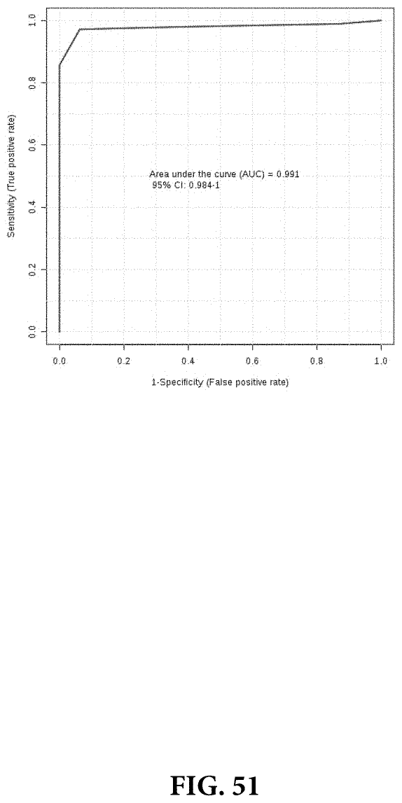

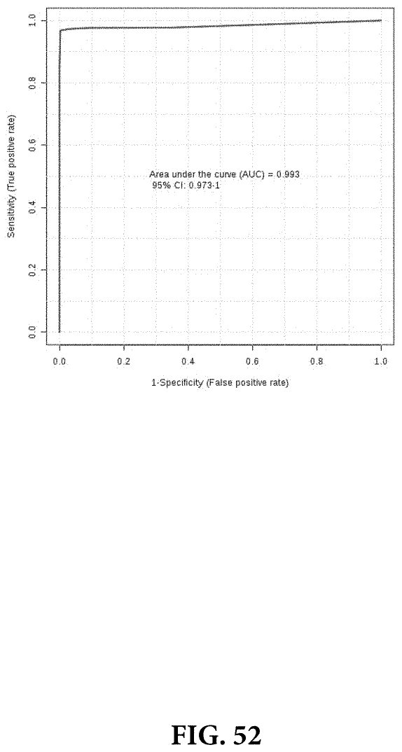

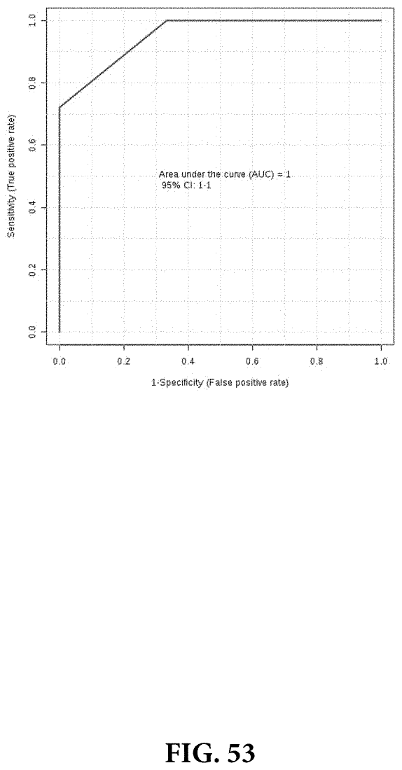

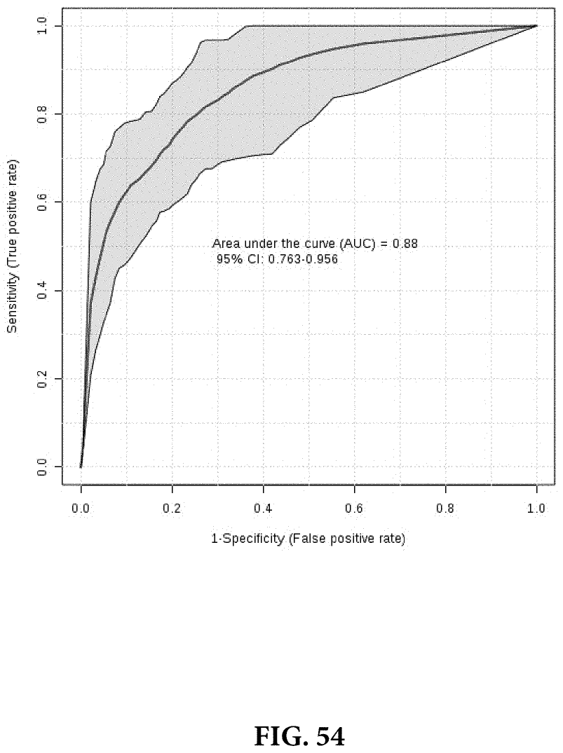

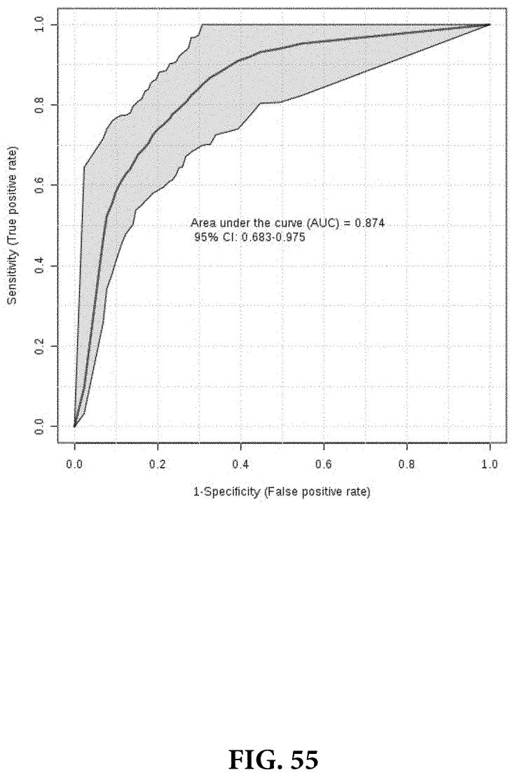

FIGS. 49-55 analyze metabolites and clinical factor contributions for diagnosing combined stage 1 (adenocarcinoma and squamous) lung cancer patients.

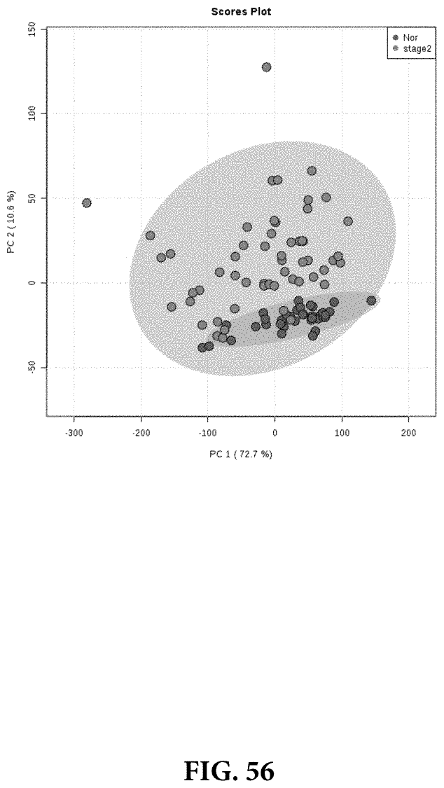

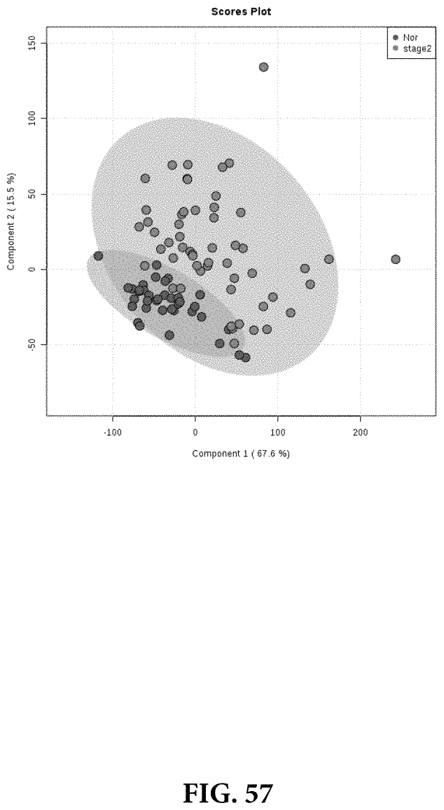

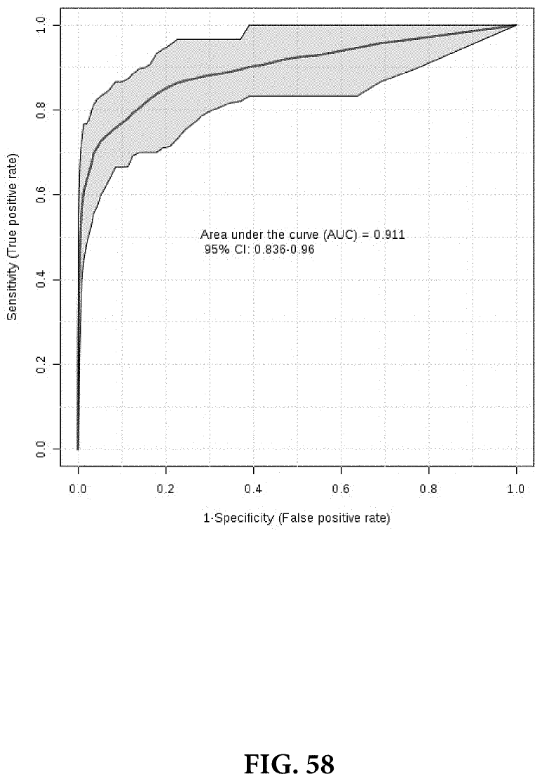

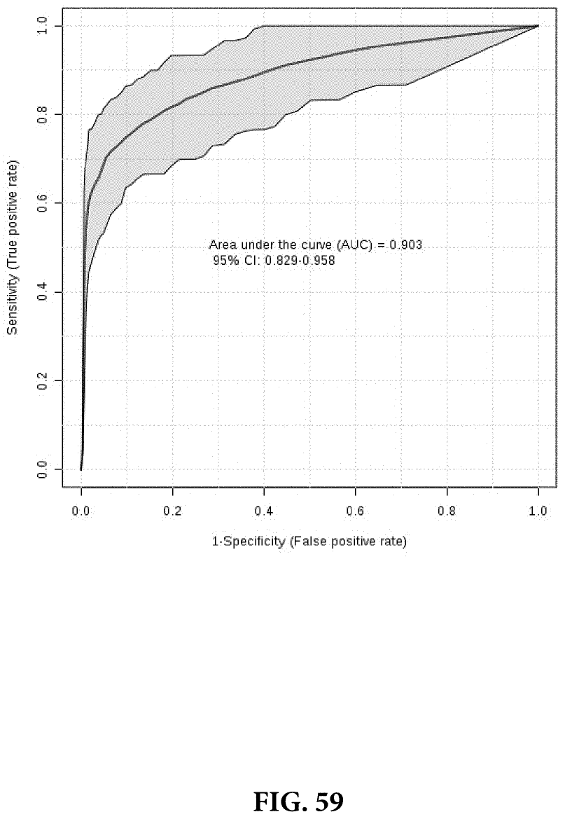

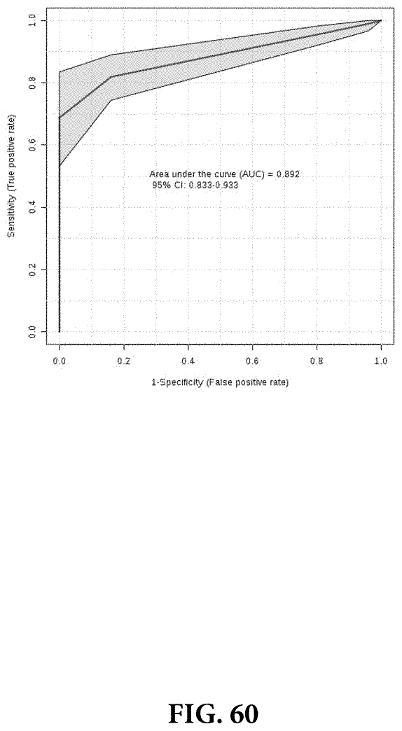

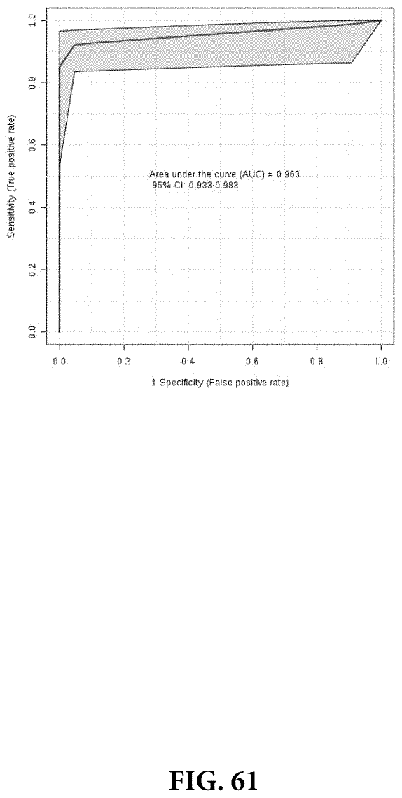

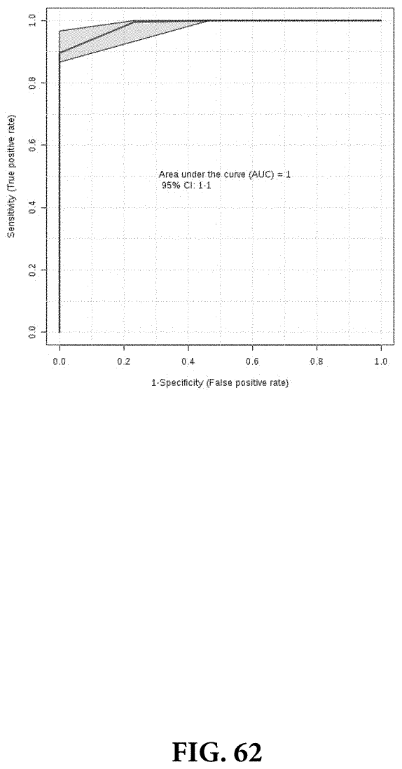

FIGS. 56-62 analyze metabolites and clinical factor contributions for diagnosing combined stage 2 (adenocarcinoma and squamous) lung cancer patients.

It will be understood by a person skilled in the art that many of the details provided above are by way of example only, and are not intended to limit the scope of the invention which is to be determined with reference to the following claims.

1. A biomarker panel for a serum test for detecting lung cancer, wherein the biomarkers are selected from the group of biomarkers consisting of arginine, C18.2, decadienylcarnitine (C10:2), LYSOC18.2, methionine, ornithine, PC32:2AA, PC36.0AA, PC36.0AE, putrescine, spermidine, spermine, and valine.

2. The biomarker panel of claim 1, wherein the serum test for diagnosing lung cancer accounts for smoking history.

3. Use of the biomarker panel of claim 1 to diagnose stage 1 lung cancer.

4. Use of the biomarker panel of claim 1 to diagnose stage 2 lung cancer.

5. Use of the biomarker panel of claim 1 to differentiate between stage 1 adenocarcinoma lung cancer and stage 1 squamous lung cancer.

6. Use of the biomarker panel of claim 1 to differentiate between stage 2 adenocarcinoma lung cancer and stage 2 squamous lung cancer.

7. Use of the biomarker panel of claim 1 to diagnose combined stage 1 and 2 adenocarcinoma lung cancer.

8. Use of the biomarker panel of claim 1 to diagnose combined stage 1 and 2 squamous lung cancer.

9. Use of the biomarker panel of claim 1 to diagnose combined stage 1 adenocarcinoma lung cancer and squamous lung cancer.

10. Use of the biomarker panel of claim 1 to diagnose combined stage 2 adenocarcinoma lung cancer and squamous lung cancer.

11. Use of the biomarker panel of claim 1 to diagnose late stage 3b/4 lung cancer.
